(19) United States
(12) Patent Application Publication
(10) Pub. No.: US 2023/0390383 A1
(43) Pub. Date: Dec. 7, 2023

(54) REPLICATION INCOMPETENT INFLUENZA VACCINE PLATFORM FOR FOREIGN PROTEIN DELIVERY

(71) Applicant: Duke University, Durham, NC (US)

(72) Inventors: Nicholas S. HEATON, Durham, NC (US); Alanson GIRTON, Frederick, MD (US); Zhaochen LUO, Durham, NC (US); Stacy WEBB, Durham, NC (US)

(21) Appl. No.: 18/033,039

(22) PCT Filed: Oct. 20, 2021
(86) PCT No.: PCT/US2021/055840

§ 371 Date: Apr. 20, 2023

Related U.S. Application Data
(60) Provisional application No. 63/093,926, filed on Oct. 20, 2020.

(51) Int. Cl. A61K 39/215; C12N 7/00

(52) U.S. Cl. CPC A61K 39/215; C12N 7/00; A61K 2039/5256

(57) ABSTRACT

The present invention provides replication incompetent influenza viral particles comprising a modified hemagglutinin (HA) protein. Also provided are methods for making and using the viral particles, and cell lines for making the viral particles.

### CROSS-REFERENCE TO RELATED APPLICATIONS

This application claims priority to U.S. Provisional Application No. 63/093,926 filed on Oct. 20, 2020, the contents of which are incorporated by reference in their entireties.

### STATEMENT REGARDING FEDERALLY SPONSORED RESEARCH

This invention was made with government support under grant number 75N93019C00050 awarded by the National Institute of Allergy and Infectious Diseases, National Institutes of Health. The government has certain rights in this invention.

### SEQUENCE LISTING

A Sequence Listing accompanies this application and is submitted as an ASCII text file of the sequence listing named “155554_00620_ST25.txt” which is 56,854 bytes in size and was created on Oct. 18, 2021. The sequence listing is electronically submitted via EFS-Web with the application and is incorporated herein by reference in its entirety.

### BACKGROUND

Infections with influenza viruses cause annual epidemics of respiratory disease, and as such, impose a large burden on human health (1). Influenza disease severity ranges from mild to severe, and it is estimated that 3 to 5 million cases of severe illness and 290,000 to 650,000 respiratory deaths worldwide are the result of influenza viral infections (2). In the United States alone, there have been between nine and 35 million cases of illness, and 140,000 to 710,000 hospitalizations annually since 2010 (3). This disease burden is in spite of FDA approved antiviral inhibitors and annual vaccination campaigns (4, 5).

Despite suboptimal efficacy, the best prophylactic measure to prevent influenza remains vaccination (6). Influenza virus vaccines currently afford short-term protection from viruses that are closely related to the vaccine strains. The seasonal influenza vaccines currently in use are predominately designed and formulated to induce antibodies against hemagglutinin (HA). This is in no small part because the hemagglutinin inhibition (HAI) titer of serum is a well-recognized correlate of protection from influenza virus infection (7). However, the antibodies elicited by current vaccines are typically against the immunodominant HA globular head domain, which is highly variable and plastic, and typically only provides strain-specific protection (8, 9). Due to the lack of strong heterologous protection, new versions of influenza vaccines are developed each year because of viral antigenic drift (10). Further, in addition to seasonal influenza, pandemic outbreaks are typically caused by antigenically distinct viruses against which seasonal vaccines are likely to provide limited protection (6).

Thus, there remains a need in the art for improved IAV vaccines that are broadly effective authentic influenza viral particles.

### SUMMARY

In a first aspect, the present invention provides modified influenza viral particles. The viral particles comprise a modified HA protein comprising the transmembrane domain of HA and the cytoplasmic tail of HA. The modification to the HA gene renders these viral particles replication incompetent.

In a second aspect, the present invention provides vaccine formulations comprising a viral particle described herein and a pharmaceutically acceptable carrier.

In a third aspect, the present invention provides methods for producing the viral particles described herein. Two different embodiments of these methods are described. In the first embodiment, the methods comprise: (a) modifying the HA gene within segment 4 of the genome of an influenza virus in a manner that renders the virus replication incompetent; (b) transfecting the modified genome into a first cell line that expresses wild-type HA on its surface; (c) culturing the transfected first cell line to produce viral particles that comprise wild-type HA and the modified segment 4 of step (a); (d) infecting a second cell line that expresses a modified HA protein comprising the transmembrane domain of HA and the cytoplasmic tail of HA with the viral particles produced in step (c); and (e) culturing the infected second cell line to produce replication incompetent viral particles that comprise the modified HA protein.

In the second embodiment, the methods comprise: (a) modifying the HA gene within segment 4 of the genome of an influenza virus to encode a modified HA protein comprising the transmembrane domain of HA and the cytoplasmic tail of HA, thereby rendering the virus replication incompetent; (b) transfecting the modified genome into a first cell line that expresses wild-type HA on its surface; (c) culturing the transfected first cell line to produce viral particles that comprise wild-type HA and the modified segment 4 of step (a); (d) infecting a second cell line that does not express HA with the viral particles produced in step (c); and (e) culturing the infected second cell line to produce replication incompetent viral particles that comprise the modified HA protein.

In a fourth aspect, the present invention provides methods for inducing an immune response in a subject. The methods comprise administering a viral particle or vaccine formulation described herein to the subject.

In a fifth aspect, the present invention provides influenza-susceptible cell lines that express a modified HA protein comprising the transmembrane domain of HA and the cytoplasmic tail of HA.

### BRIEF DESCRIPTION OF THE DRAWINGS

FIG. 1 shows the generation of hemagglutinin (HA)-negative influenza A virus (IAV) particles. (A) Left: Typical reproduction cycle of IAV. Middle: Reproduction cycle of an IAV in which the HA segment has been replaced with mCherry. Viral replication can occur on cells that stably express the IAV HA protein on the cell surface, which is subsequently packaged into progeny virions. Right: Reproduction of mCherry IAV in cells that express the transmembrane and cytoplasmic domains of HA fused to GFP in place of the normal HA ectodomain (HAₜₘ-GFP). Progeny virions produced by these cells are unable to subsequently infect cells. (B) Immunofluorescence microscopy of HA-MDCK cells infected with segment 4 IAV, M01=0.1, scale bar=100 m. (C) Immunofluorescence microscopy of HAtm-GFP MDCK cells infected with segment 4 IAV, MOI=10, scale bar=100 m. (D) Western blots of proteins from the indicated, purified and concentrated viral particles. (E) Densitometric analysis of NA from western blot, each dot represents a repeat. (F) Densitometric analysis of the M2 from western blot, each dot represents a repeat. Error bars represent the SD. Statistical differences were determined using an unpaired Student's t-test and are denoted as follows: *P<0.05

FIG. 2 shows sera reactivity against the vaccine matched strain, A/Puerto Rico/8/1934. Mice were vaccinated intramuscularly with BSA, inactivated WT PR8 IAV, or HA-negative PR8 IAV. Animals then received a single boost between 14 and 21 days later. 7-14 days after the boost, sera were collected and analyzed for antigen reactive antibodies (BSA, WT IAV, HA-negative groups, n=5; Blank, n≥2). (A) Serum reactivity against intact, whole PR8 viral particles. (B) Area under the curve analysis of A. (C) Serum reactivity against the PR8 HA protein. (D) Area under the curve analysis of C. (E) Serum hemagglutination inhibition (HAI) antibody response to PR8. ND indicates that the samples were below the limit of detection (LOD). For statistical analysis of undetected samples, a value of one half of the LOD was used. (F) Serum reactivity against the PR8 NA protein. (G) Area under the curve analysis of F. (H) Serum reactivity against the PR8 M2 protein. (I) Area under the curve analysis of H. Statistical differences for panel E were determined using an unpaired Student's t-test and for panels B, D, G, and I, a one-way ANOVA followed by Tukey's post-hoc analysis was used. For all panels, error bars represent the SEM and *=P<0.05; **=P<0.001; ns=not significant.

FIG. 3 shows the results of homologous viral challenge after WT or HA-negative IAV vaccination. (A) Diagram of the immunization scheme and timepoints for sample collection. (B-C) Mice body weight (B) and survival (C) after intranasal infection with PR8 (n=5). In panel B, * indicates p<0.001 comparing HA-negative IAV and BSA treatment groups. ** indicates p<0.001 comparing the BSA group to both virally vaccinated groups. In panel C, ** indicates p<0.001 between both virally vaccinated groups and the BSA control group. Bodyweight changes and survival after viral challenge were analyzed by a two-way ANOVA followed by a Sidak's multiple comparisons test or a log-rank (Mantel-Cox) test, respectively. (D) Virus titers in lung tissue after intranasal PR8 infection (n=5). Statistical differences were determined via a one-way ANOVA followed by Tukey's post-hoc analysis. ND indicates the samples were below the limit of detection (LOD). For statistical analysis of undetected samples, a value of one half of the LOD was used. (E) Lung tissue histology after mice intranasally infected with PR8 virus, scale bar=50 m. For all panels, error bars represent the SEM. Statistical differences are denoted as follows: **P<0.001; ns=not significant.

FIG. 4 shows the reactivity of PR8-based vaccination derived sera against the heterologous strain, A/California/04/09. Mice were primed intramuscularly with BSA, WT PR8 IAV or HA-negative PR8 IAV and received a single boost two to three weeks later (BSA, WT IAV, HA-negative groups, n=5; Blank, n=3). (A) Sera reactivity against intact whole Cal09 virus. (B) Area under the curve analysis of A. (C) Sera reactivity against the Cal09 HA protein. (D) Area under the curve analysis of C. (E) Sera reactivity against the Cal09 NA protein. (F) Area under the curve analysis of E. (G) Serum hemagglutination inhibition (HAI) antibody response to Cal09. Statistical differences for panels B, D, and F were determined via one-way ANOVA followed by Tukey's post-hoc analysis. For all panels, error bars represent the SEM and *=P<0.05; **=P<0.001; ns=not significant. ND indicates the samples were below the limit of detection.

FIG. 5 shows the results of heterologous viral challenge after WT or HA-negative IAV vaccination. (A) Diagram of the immunization scheme and timepoints for sample collection. The WT IAV and HA-negative IAV vaccines were PR8 based, the same as in FIG. 4. (B-C) Mice body weight (B) and survival (C) after intranasal infection with Cal09 (n=5). In panel B, * indicates p<0.05 comparing the WT IAV and BSA groups and p<0.001 comparing the HA-negative IAV and BSA treatment groups. ** indicates p<0.001 comparing the BSA group to both virally vaccinated groups. In panel C, ** indicates p<0.001 between both vaccine groups and the BSA control group. Bodyweight changes and survival after viral challenge were analyzed by a two-way ANOVA followed by a Sidak's multiple comparisons test or a log-rank (Mantel-Cox) test, respectively. (D) Virus titers in lung tissue after intranasal Cal09 infection (n=5). Statistical differences were determined via a one-way ANOVA followed by Tukey's post-hoc analysis. (E) Lung tissue histology after mice intranasally infected with Cal09 virus, scale bar=50 m. For all panels, error bars represent the SEM. Statistical differences are denoted as follows: *P<0.05; **=P<0.001; ns=not significant.

FIG. 6 shows the nucleotide sequence of the fusion protein comprising GFP and the IAV HA transmembrane domain and cytoplasmic tail.

FIG. 7 shows the generation of HA-negative IAV particles that comprise a heterologous viral antigen. (A) Left: Typical reproduction cycle of IAV. Middle: Reproduction cycle of an IAV in which the HA segment has been replaced with mCherry. Viral replication can occur on cells that stably express the IAV HA protein on the cell surface, which is subsequently packaged into progeny virions. Right: Reproduction of mCherry IAV in cells express the transmembrane and cytoplasmic domains of HA fused to a heterologous antigen (i.e., the receptor binding domain (RBD) of the spike protein of SARS-CoV-2) in place of the normal HA ectodomain (HAₜₘ-RBD). Progeny virions produced by these cells are unable to subsequently infect cells. (B) Shows MDCK cells expressing HAₜₘ-RBD stained with anti-SARS-CoV-2 RBD antibody.

FIG. 8 shows the generation of HA-negative IAV particles that express a heterologous viral antigen from segment 4 of the IAV genome. (A) Left: Typical reproduction cycle of IAV. Middle: Reproduction cycle of an IAV in which the HA segment has been replaced with a heterologous antigen. Viral replication can occur on cells that stably express the IAV HA protein on the cell surface, which is subsequently packaged into progeny virions. Right: Production of the modified IAV in cells do not express HA. Progeny virions produced by these cells are unable to subsequently infect cells. The heterologous antigen is present on these viruses when grown under terminal and non-terminal growth conditions. (B) Schematic illustrating genetic manipulations of segment 4 to encode a heterologous antigen (SARS-CoV-2 RBD) in several different ways. (C) Hemagglutinin assay showing the presence of virus after successfully rescuing virus from DNA in 293T cells. (D) HA units of rescued viruses (shows total number of particles) compared to wild-type virus (PR8). (E) Quantified viral titers from virus rescues (PFU, plaque forming units) compared to PR8. (F) Whole virus enzyme-linked immunosorbent assay (ELISA) using an anti-SARS-CoV-2 RBD antibody.

FIG. 9 shows the nucleotide and amino acid sequences of the segment 4 heterologous antigen (SARS-CoV-2 RBD) constructs depicted in FIG. 8B. The functional elements of the sequences are color coded as follows: HA signal peptide (blue), IL12 signal peptide (yellow), sfGFP (green), 2A cleavage site (grey), SARS-CoV-2 RBD (red), and HA transmembrane domain (purple).

FIG. 10 shows amino acid sequences of the headless HA (4G, Mini, GCN4, 6SS) designs aligned to wild-type HA.

FIG. 11 shows headless HA design validation. (A) 293T cells were transfected with the designed headless HA expression plasmids, and then stained with primary antibodies that specifically recognize the head (PY102) or stalk (6F12, CR6261, CR9914) of hemagglutinin. Samples were then stained with secondary antibody conjugated to AlexaFluor-488. Flow cytometry was used to measure primary antibody binding. Wild-type HA was used as a positive control. Of the 4 headless HA designs, only cells expressing the 6SS design were positive for stalk antibody binding and negative for head antibody binding. (B) Schematic of the 6SS headless HA design, along with the nucleotide and amino acid sequences.

FIG. 12 shows the generation of IAV particles that comprise a headless HA (hlHA) protein. (A) Schematic of headless HA virus propagation strategy. 293T cells are transfected with wild-type HA expression plasmid and then infected with an influenza virus encoding mCherry in place of HA in segment 4 (PR8-deltaHA-mCherry). Supernatant containing the newly propagated virus (PR8-deltaHA-mCherry) is then placed on 293T cells transfected with the 6SS headless HA expression construct. The supernatant contains virus expressing the headless HA (PR8-deltaHA-mCherry-6SShlHA). (B) Wild-type and 6SS headless HA viruses were concentrated and western blotting was used to detect viral protein expression. HA protein can be detected in concentrated wild-type virus but not the 6SS headless virus. Both the M and NA proteins can be detected for both viruses.

FIG. 13 demonstrates that mice vaccinated with 6SS generate higher antibody responses against hlHA, NA only viruses. Mice were vaccinated with inactivated wild-type PR8, 6SS headless HA PR8, a PR8 virus with surface expression of GFP in place of HA, or a BSA control. ELISA was used to measure serum reactivity against 6SS headless HA virus (left) and a virus that has all viral proteins except HA (right) as a proof of concept.

FIG. 14 demonstrates generation of stable cell line expressing hlHA for virus propagation. (A) MDCK cells were transduced with lentivirus packaging the 6SS headless HA construct to generate cell lines with stable expression of headless HA for virus propagation. For validation, 6SS headless HA MDCK cells along with wild-type MDCK cells and MDCK cells stably expressing wild-type PR8 HA were stained with primary antibodies targeting the head or stalk of hemagglutinin. Samples were then stained with secondary antibody conjugated to AF488 and flow cytometry was used to measure primary antibody binding. A population of cells in both headless HA MDCK cell lines positive for stalk staining. (B,C) FACS was used to collect MDCK cells with high expression of 6SS headless HA (based on antibody staining). The collected cells were expanded, and flow cytometry was used to determine the percentage of cells with expression of 6SS headless HA. MDCK cells expressing wild-type PR8 HA or GFP were used as a control.

FIG. 15 shows the generation of IAV particles that comprise hlHA via propagation on hlHA-MDCK cells. MDCK cells with stable expression of PR8 HA are infected with an influenza virus encoding mCherry in place of HA in segment 4 (PR8-deltaHA-mCherry). Supernatant containing the newly propagated virus (PR8-deltaHA-mCherry) is then placed on MDCK cells with stable expression of 6SS headless HA. The supernatant contains virus expressing the headless HA (PR8-deltaHA-mCherry-6SShlHA).

### DETAILED DESCRIPTION

The present invention provides replication incompetent influenza viral particles comprising a modified hemagglutinin (HA) protein. Also provided are methods for making and using the viral particles, and cell lines for making the viral particles.

To better combat seasonal influenza, and prepare for pandemic influenza viruses of unknown antigenicity, novel vaccines that induce cross-protective immunity against diverse influenza viruses are highly desirable. Theoretically, a “universal” influenza vaccine would elicit broadly protective immune responses by redirecting immune responses to more highly conserved viral epitopes (11). While the relatively conserved hemagglutinin (HA) “stalk” domain has long been one such a target (12-14), it is known that immunity against other viral structural proteins can also contribute to protection. For example, neuraminidase (NA) is the second most abundant glycoprotein on the surface of virions and can evolve independently of HA (15, 16), suggesting that anti-neuraminidase immunity may be able to afford protection even when the HA protein is highly drifted. Accordingly, serum neuraminidase inhibition (NI) or anti-NA antibodies are correlated with decreased susceptibility to heterologous influenza strains (17-19). The extracellular domain of the M2 protein (M2e) is also well conserved among different human influenza A virus strains (20). Previous studies have demonstrated that M2e-containing virus-like particles (VLPs) or vectored M2e vaccines could induce broad cross-reactive immune responses and provide protection against heterologous and heterosubtypic challenge in mice (21, 22). Nucleoprotein (NP) and matrix protein 1 (M1) are internal proteins that are highly conserved between all influenza A subtypes. Vaccines containing NP alone or in combination with M1 have been reported to induce a cross-protective T-cell response against influenza viruses of different subtypes (23, 24).

While it is clear that non-HA structural proteins can contribute to vaccine-mediated protection from influenza disease, the vast majority of studies have taken reductionist approaches and evaluated the antigens outside of the context of the other viral proteins. In contrast, the present inventors have taken a “subtractive” approach and have generated authentic influenza viral particles that contain all the viral proteins with the exception of the HA protein. To generate these viral particles, they genetically eliminated the HA open reading frame (ORF) from the influenza A virus (IAV) genome. By performing viral propagation on two different helper cell lines, they were able to produce IAV viral particles that lack the HA protein. They then used these “HA-negative” viruses to probe the nature of the immunity elicited by HA-containing or HA-negative inactivated viral vaccination. Specifically, the HA-negative viral particles allowed the inventors to evaluate the contributions of all the non-HA antigens to protection from viral challenge at the same time. They found that, while HA-based immunity was a significant contributor to protection against a homologous viral strain (i.e., a vaccine-matched strain), there was no significant difference in protection against a heterologous viral strain (i.e., H1N1). Their work supports the importance of including non-HA structural proteins in universal influenza vaccines.

### Viral Particles:

In a first aspect, the present invention provides modified influenza viral particles. The viral particles comprise a modified HA protein comprising the transmembrane domain of HA and the cytoplasmic tail of HA. These viral particles are missing the head domain of HA which is immunodominant in natural infection, is subject to antigenic drift and mediates viral entry. Importantly, the modification to the HA gene renders these viral particles replication incompetent.

The influenza virus is a negative-sense, single-stranded RNA virus. Influenza viruses can be divided into four distinct subtypes (influenza A, influenza B, influenza C, and influenza D) based on their nucleoproteins and the antigen determinants of their matrix proteins. Human influenza A and B viruses are responsible for the seasonal flu. Thus, the modified viral particles of the present invention may be derived from either influenza A or influenza B.

The terms “viral particle” and “virion” are used interchangeably herein to refer to the extracellular phase of a virus. An influenza viral particle consists of a nucleic acid core (i.e., the viral genome), an outer protein coating or capsid, and an outer envelope made of protein and phospholipid membrane derived from the host cell that produced the viral particle. The genome of influenza A and influenza B viruses are segmented into eight separate strands.

Hemagglutinin (HA) is a glycoprotein found on the surface of influenza viral particles. The HA protein used with the present invention may be of any subtype including, without limitation, H1 through H18. Suitably, the HA protein may be an H1, H2, H3, or H5 subtype. The HA protein is a homotrimer where each monomer is a single polypeptide chain having an HA1 and HA2 region. The HA2 region sits on top of the HA1 region. The HA1 comprises the head domain which comprises the cell binding region and is immunodominant. The HA1 and HA2 regions are linked by disulfide bridges. The headless HA provided herein lacks a portion of HA1. The virus particle having the HA stalk domain lacks the head region. See Steel et al. 2010. An influenza virus vaccine based on the Conserved Hemagglutinin Stalk Domain. mBio 1(1):e00018-10.

As used herein, a “wild-type HA protein” is an HA protein that is in its natural, unmodified form. In contrast to the modified HA proteins described herein, a wild-type HA protein has the ability to promote viral entry into a cell. Specifically, a wild-type HA protein has the ability to bind to sialic acid-containing receptors on the surface of the cell and promote fusion of the viral membrane with the cell membrane. An exemplary wild-type HA protein sequence is provided as SEQ ID NO:18. However, the sequences of other wild-type HA proteins are known in the art and may be used in place of this sequence.

A “modified HA protein” is encoded by an HA gene that has been genetically modified to reduce or eliminate the ability of the HA protein to promote viral entry into a cell. Importantly, this modification of the HA gene must render the viral particles replication incompetent. Suitable genetic modifications that can be used to disrupt HA protein function include deletions, insertions, amino acid substitutions, and integrations of exogenous DNA.

As used herein, the term “replication incompetent” is used to describe viruses that are defective for one or more functions that are essential for viral genome replication or synthesis and assembly of viral particles. The virus particles of the present invention are replication incompetent because they do not comprise a fully functional (e.g., wild-type) HA protein.

The modified HA proteins used with the present invention comprise the transmembrane domain of HA and the cytoplasmic tail of HA. In some embodiments, the amino acid sequence encoding the transmembrane domain and cytoplasmic tail of HA is SEQ ID NO:10 or a polypeptide having at least 70%, 75%, 80%, 85%, 90%, 95%, or 99% sequence identity to SEQ ID NO:10. In some embodiments, the transmembrane domain and cytoplasmic tail are the only HA protein domains that are included in the modified HA protein. In other embodiments, the modified HA protein comprises most of or all of the HA protein domains but comprises a disabling mutation. In some embodiments the HA is a headless HA in which the head region of the HA is removed.

In some embodiments, the modified HA protein further comprises the stalk domain of HA, such that the stalk domain is present on the surface of the viral particle. Because the stalk domain of HA is highly conserved, it has great potential for use as an antigen in a universal vaccine that provides broad cross-protection against different influenza subtypes. In specific embodiments, the amino acid sequence encoding the stalk domain is SEQ ID NO:24 or SEQ ID NO:25 or a polypeptide having at least 70%, 75%, 80%, 85%, 90%, 95%, or 99% sequence identity to SEQ ID NO:24 or 25. SEQ ID NO:24 is the sequence of the stalk domain found in the wild-type HA protein. Notably, the sequence of the head domain (SEQ ID NO: 26) is inserted within the stalk domain within the full-length wild-type HA protein (SEQ ID NO:18). SEQ ID NO:25 is the sequence of the stalk domain found in the 6SS headless HA protein. In the 6SS stalk domain, the HA1 sequence is replaced with a -GSG- linker and a loop on the stalk is replaced with a -GSGGSG- linker (SEQ ID NO:28). Thus, the 6SS stalk domain does not comprise the full-length HA stalk domain.

In some embodiments, the modified HA protein is a headless HA protein, as described in Example 3 or an HA lacking at least a portion of the head domain of the HA protein. A “headless HA protein” is an HA protein that lacks the globular head domain of HA (e.g., SEQ ID NO:26). The head domain of HA is immunodominant, meaning that the immune response to the HA protein is skewed in favor of epitopes within this domain. Thus, elimination of the head domain from the HA protein allows for the generation of HA proteins with altered immunogenicities. For example, elimination of the head domain may generate HA proteins in which epitopes that are typically subdominant (i.e., epitopes that are not targeted or targeted to a lower degree during an immune response), such as the HA stalk domain, become immunodominant. Suitable headless HA proteins include those disclosed as SEQ ID NOs:19-22 or a polypeptide having at least 70%, 75%, 80%, 85%, 90%, 95%, or 99% sequence identity to SEQ ID NO:19-22. In SEQ ID NO:19, referred to herein as 4G headless HA, the HA1 sequences between Cys52 and Cys277 is replaced with a -GGGG- linker (SEQ ID NO:27). In SEQ ID NOs:20 and 21, referred to herein as mini headless HA and GCN4 headless HA, respectively, a majority of the HA1 sequence is replaced with a -GGGG- linker (SEQ ID NO:27) and a disulfide bond is introduced to stabilize the HA2 trimers. Mini headless HA does not include the trimerization motif (GCN4), whereas GCN4 headless HA does. In SEQ ID NO:22, referred to herein as 6SS headless HA, the HA1 sequence is replaced with a -GSG- linker and a loop on the stalk is replaced with a -GSGGSG- linker (SEQ ID NO:28). Of these four headless HA designs, 6SS is the only headless HA that is thought to fold correctly based on the ability of the stalk-specific antibody 6F12 to bind to it. Thus, in preferred embodiments, the headless HA protein is that of SEQ ID NO:22 (i.e., the 6SS headless HA). Those of skill in the art can design other HA proteins lacking the ability to bind to and allow replication of the virus and lacking immunodominant epitopes for use in the viral particles and methods described herein. The included HA proteins may be described as “HA proteins with altered immunogenicities” in which immunodominant epitopes are eliminated from the HA. These immunodominant epitopes are often not highly conserved and are susceptible to antigenic drift. The Has designed herein would allow targeting of the immune response to more conserved epitopes to generate a broad-spectrum vaccine. In one embodiment, the modified HA comprises 99 nucleotides at the 5′ end of the protein (33 N-terminal amino acids) and 150 nucleotides at the 3′ end of the gene (50 amino acids at the C-terminal end of the protein). The 3′ terminal nucleotides may be further modified such that any ATG codons are modified to TTG codons to avoid translation defects and obtain expression of the modified HA.

In some embodiments, the modified HA protein further comprises a heterologous protein that is present on the surface of the viral particle. As used herein, a “heterologous protein” refers to a protein that is not found in an influenza virus in nature (i.e. non-native). Suitable heterologous proteins include, without limitation, fluorescent proteins and antigenic proteins. A “fluorescent protein” is any protein that emits light when exposed to light. Exemplary fluorescent proteins include, without limitation, zsGreen, mRuby, mCherry, green fluorescent proteins (GFPs) and GFP variants (e.g., sfGFP), yellow fluorescent proteins (YFPs), red fluorescent proteins (RFPs), DsRed fluorescent proteins, far-red fluorescent proteins, orange fluorescent proteins (OFPs), blue fluorescent proteins (BFPs), cyan fluorescent protein (CFPs), Kindling red protein, and JRed. An “antigenic protein” is a protein that can serve as an antigen (i.e., a substance that induces an immune response). Suitable antigenic polypeptides may include, without limitation, viral antigens, bacterial antigens, fungal antigens, parasitic antigens and tumor-specific antigens.

In some embodiments, the heterologous protein is a viral antigen. Suitable viral antigens include proteins produced by viruses such as coronaviruses, alphaviruses, flaviviruses, adenoviruses, herpesviruses, poxviruses, parvoviruses, reoviruses, picornaviruses, togaviruses, orthomyxoviruses, rhabdoviruses, retroviruses, hepadnaviruses, herpesviruses, rhinoviruses, cytomegalovirus, Karposi sarcoma virus, human papillomavirus (HPV), human immunodeficiency virus (HIV), herpes simplex virus, herpesvirus 1, herpesvirus 2, herpesvirus 6, herpesvirus 7, herpesvirus 8, hepatitis A, hepatitis B, hepatitis C, measles, mumps, parvovirus, rabies virus, rubella virus, varicella zoster virus, ebola virus, west niles virus, yellow fever virus, dengue virus, rotovirus, zika virus, and the like.

In some embodiments, the viral antigen is from severe acute respiratory syndrome coronavirus 2 (SARS-CoV-2). Suitable SARS-CoV-2 antigens include, without limitation, those derived from the spike (S), nucleocapsid (N), envelope (E), and membrane (M) structural proteins. In some embodiments, the viral antigen is the receptor binding domain (RBD) of the spike protein from SARS-CoV-2 (SARS-CoV-2 RBD). In specific embodiments, the amino acid sequence encoding the SARS-CoV-2 RBD is SEQ ID NO:11 or a polypeptide having at least 70%, 75%, 80%, 85%, 90%, 95%, or 99% sequence identity to SEQ ID NO:11.

The heterologous protein is localized to the surface of the viral particles via its expression as a fusion protein with the modified HA protein, which comprises the transmembrane domain of HA and the cytoplasmic tail of HA. The transmembrane domain of HA anchors the fusion protein in the cell membrane, such that the heterologous protein can be expressed on the cell surface. In some embodiments, the C-terminal end of the heterologous protein is fused to the N-terminal end of the transmembrane domain of HA within the fusion protein.

To ensure that the heterologous protein is present on the surface of the viral particle, the modified HA protein may include a signal peptide at the N-terminus for membrane trafficking. In some embodiments, the signal peptide is an HA signal peptide. The HA signal peptide may include the polypeptide of SEQ ID NO:14 or a polypeptide having at least 70%, 75%, 80%, 85%, 90%, 95%, or 99% sequence identity to SEQ ID NO:14. In other embodiments, the signal peptide is an IL12 signal peptide, which has been well characterized and is efficiently targeted to the cell membrane. The IL12 signal peptide may include the polypeptide of SEQ ID NO:15 or a polypeptide having at least 70%, 75%, 80%, 85%, 90%, 95%, or 99% sequence identity to SEQ ID NO:15. However, any signal peptide that targets a protein to the cellular membrane may be used in the modified HA protein.

In some embodiments, the modified HA protein comprises one or more linker peptides. As used herein, the term “linker peptide” refers to a peptide sequence that bridges two protein components within a fusion protein. The linker may be an existing portion of a protein component included in the fusion protein or it may be provided by insertion of one or more amino acid residues between the protein components of the fusion protein. In some embodiments, the linker peptide is a -GGGG- linker (SEQ ID NO:27), a -GSG- linker, or a -GSGGSG- linker (SEQ ID NO:28). In some embodiments, the linker peptide is a “detachable linker”, i.e., a linker that results in the separation of the protein components flanking the linker. In some embodiments, the detachable linker is a self-cleaving 2A polypeptide. Self-cleaving 2A polypeptides are known in the art as described, for example, in Kim, J. H. et al., PLOS ONE, 6(4), e18556. Suitable self-cleaving 2A polypeptides may include, without limitation, FMDV 2A, equine rhinitis A virus (ERAV) 2A (E2A), porcine teschovirus-1 2A (PTV1-2A), and Thoseaasigna virus 2A (T2A). In some embodiments, the self-cleaving 2A polypeptide comprises SEQ ID NO:16 or a polypeptide having at least 70%, 75%, 80%, 85%, 90%, 95%, or 99% sequence identity to SEQ ID NO:16.

In some embodiments, the modified HA protein is derived from the host cell that produced the viral particle and is not encoded in the viral genome. In other embodiments, the modified HA protein is encoded in the viral genome, preferably in segment 4.

In embodiments in which the HA protein is encoded in the viral genome, the gene encoding the modified HA protein may further include additional polynucleotides typically found the influenza genome, such as an influenza virus packaging signal. As used herein, an “influenza virus packaging signal” refers to any cis-acting sequence or sequences that are required to ensure that each influenza virion has a full complement of the influenza genome. Influenza virus packaging signal(s) have been identified for each influenza A virus segment (see, e.g., Gao et al., J. Virol. 86:7043-7051 (2012)). A suitable influenza virus packaging signal may include, without limitation, SEQ ID NO:12 and SEQ ID NO:13. In some embodiments, the modified HA genes described herein are flanked by appropriate influenza virus packaging signals within segment 4 of the viral genome. For example, the modified HA genes may be flanked at the 5′ end by the polynucleotide of SEQ ID NO:12 or a polynucleotide having at least 60%, 65%, 70%, 75%, 80%, 85%, 90%, 95%, 98%, or 99% sequence identity to SEQ ID NO:12, and may flanked at the 3′ end by the polynucleotide of SEQ ID NO:13 or a polynucleotide having at least 60%, 65%, 70%, 75%, 80%, 85%, 90%, 95%, 98%, or 99% sequence identity to SEQ ID NO:13.

The terms “protein”, “polypeptide”, and “peptide” are used interchangeably herein to refer to a polymer of amino acids. A “protein” typically comprises a polymer of naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine).

### Vaccine Formulations:

In a second aspect, the present invention provides vaccine formulations comprising a viral particle described herein and a pharmaceutically acceptable carrier.

Pharmaceutically acceptable carriers are known in the art and include, but are not limited to, diluents (e.g., Tris-HCl, acetate, phosphate), preservatives (e.g., Thimerosal, benzyl alcohol, parabens), solubilizing agents (e.g., glycerol, polyethylene glycerol), emulsifiers, liposomes, and nanoparticles. Pharmaceutically acceptable carriers may be aqueous or non-aqueous solutions, suspensions, or emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions, or suspensions, including saline and buffered media.

The vaccine formulations of the present invention may further include additives such as albumin or gelatin to prevent absorption to surfaces, detergents (e.g., Tween 20, Tween 80, Pluronic F68, bile acid salts), anti-oxidants (e.g., ascorbic acid, sodium metabisulfite), bulking substances or tonicity modifiers (e.g., lactose, mannitol). Components of the compositions may be covalently attached to polymers (e.g., polyethylene glycol), complexed with metal ions, or incorporated into or onto particulate preparations of polymeric compounds (e.g., polylactic acid, polyglycolic acid, hydrogels, etc.) or onto liposomes, microemulsions, micelles, milamellar or multilamellar vesicles, erythrocyte ghosts, or spheroplasts. The compositions may also be formulated in lipophilic depots (e.g., fatty acids, waxes, oils) for controlled or sustained release.

The vaccine formulations may also include adjuvants to increase their immunogenicity. Suitable adjuvants include, without limitation, mineral salt adjuvants, gel-based adjuvants, carbohydrate adjuvants, cytokines, or other immunostimulatory molecules. Exemplary mineral salt adjuvants include aluminum adjuvants, salts of calcium (e.g. calcium phosphate), iron, and zirconium. Exemplary gel-based adjuvants include aluminum gel-based adjuvants and acemannan. Exemplary carbohydrate adjuvants include inulin-derived adjuvants (e.g., gamma inulin, algammulin) and polysaccharides based on glucose and mannose (e.g., glucans, dextrans, lentinans, glucomannans, galactomannans). Exemplary cytokines include IFN-γ, granulocyte-macrophage colony stimulating factor (GM-CSF), IL-2, and IL-12. Suitable adjuvants also include any FDA-approved adjuvants for influenza vaccine usage including, without limitation, aluminum salt (alum) and the squalene oil-in-water emulsion systems MF59 (Wadman 2005 (Novartis)) and AS03 (GlaxoSmithKline).

In some embodiments, the vaccine formulations include a concentration of total non-infectious viral particles of at least 10⁶ pfu/mL, at least 10⁷ pfu/mL, at least 10⁸ pfu/mL, at least 10⁹ pfu/mL, at least 10¹⁰ pfu/mL, or at least 10¹¹ pfu/mL. For replication incompetent viruses the amount of virus may be based on total protein content of the viral particles or based on a single protein used as a normalization control such as based on amount or activity of neuraminidase (NA), M1 or M2.

### Methods for Producing the Viral Particles:

In a third aspect, the present invention provides methods for producing the viral particles described herein. Two different embodiments of these methods are described.

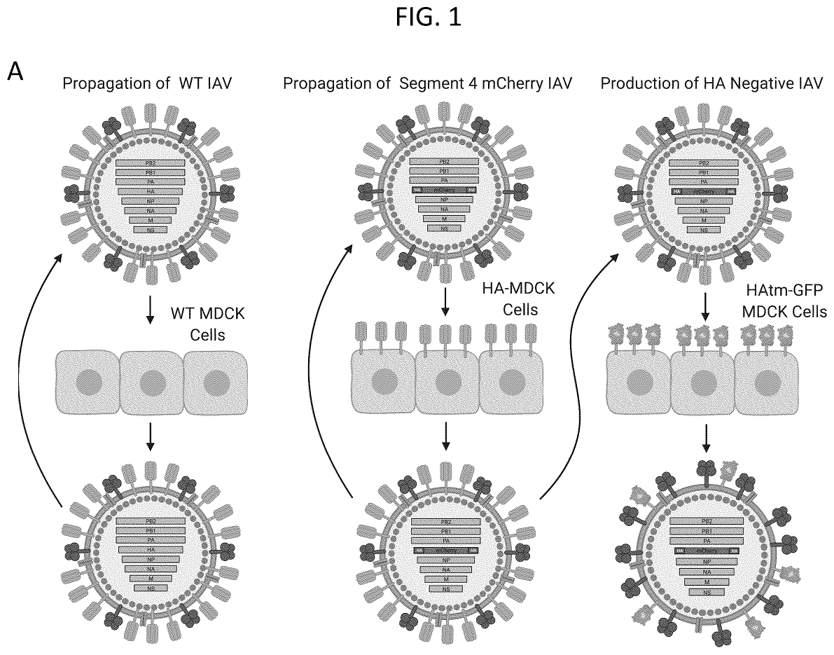

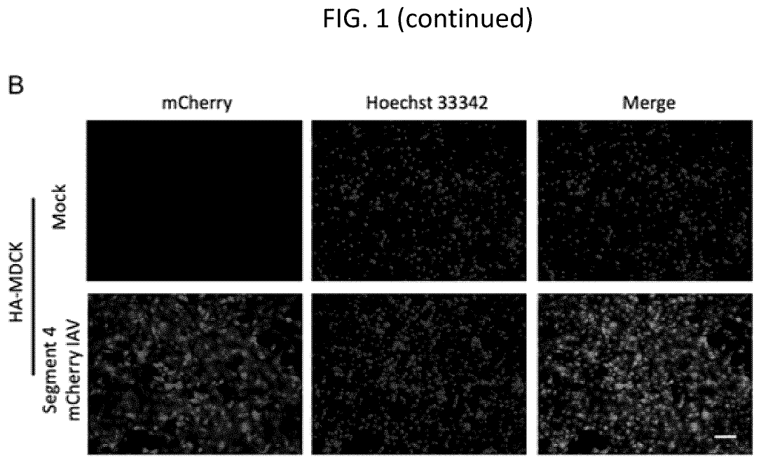

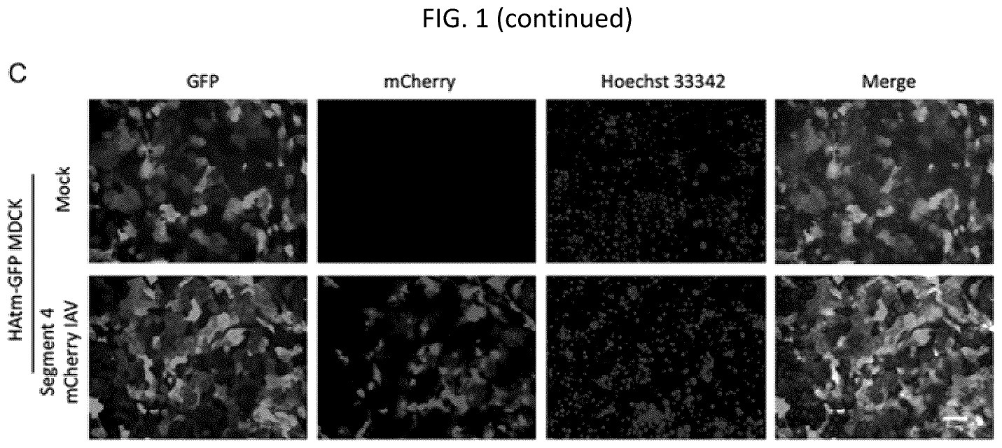

Embodiment 1: In a first embodiment, depicted in FIGS. 1A, 7A, 12A, 15, the methods comprise: (a) modifying the HA gene within segment 4 of the genome of an influenza virus in a manner that renders the virus replication incompetent; (b) transfecting the modified genome into a first cell line that expresses wild-type HA on its surface; (c) culturing the transfected first cell line to produce viral particles that comprise wild-type HA and the modified segment 4 of step (a); (d) infecting a second cell line that expresses a modified HA protein comprising the transmembrane domain of HA and the cytoplasmic tail of HA with the viral particles produced in step (c); and (e) culturing the infected second cell line to produce replication incompetent viral particles that comprise the modified HA protein.

The genome of influenza A and B viruses contains eight segments of single-stranded RNA that encode 1-2 proteins. The HA protein is encoded in segment 4. Thus, the present methods involve modifying the portion of segment 4 encoding the HA protein in a manner that renders the virus replication incompetent. In Embodiment 1, the modification of the HA gene may involve deleting a portion of the HA gene, deleting the entirety of the HA gene, introducing a mutation that prevents expression of the HA protein, introducing a mutation that results in expression of a nonfunctional HA protein, or replacing the HA gene with exogenous DNA.

As used herein, the terms “transfecting” and “transfection” refer to a process of artificially introducing nucleic acids (DNA or RNA) into cells. Transfection may be performed under natural or artificial conditions. Suitable transfection methods include, without limitation, lipofection, bacteriophage or viral infection, electroporation, heat shock, microinjection, and particle bombardment.

As used herein, the terms “infecting” and “infection” refer to a process of introducing a virus into a cell. Cells may be infected with a virus by simply contacting the cell with viral particles.

The cell lines used in the present methods are eukaryotic cell lines. Suitable eukaryotic cells include, without limitation, mammalian cells or chicken cells. The cell may be a cell in culture or may be an embryonated chicken egg. Suitable mammalian cells include, without limitation, a MDCK cell, A549 cell, a CHO cell, a HEK293 cell, a HEK293T cell, a HeLa cell, a NS0 cell, a Sp2/0 cell, a COS cell, a BK cell, a NIH3T3 cell, a FRhL-2 cell, a MRC-5 cell, a WI-38 cell, a CEF cell, a CEK cell, a DF-1 cell, or a Vero cell.

The methods for producing viral particles may further include additional steps that involve harvesting the influenza virus from the cell. In embodiments that utilize cultured cells, the methods may further comprise harvesting the supernatant of the culture by, for example, centrifugation or pipetting. In embodiments in which the cell is an embryonated chicken egg, the methods may further include harvesting the allantoic fluid from the embryonated chicken egg.

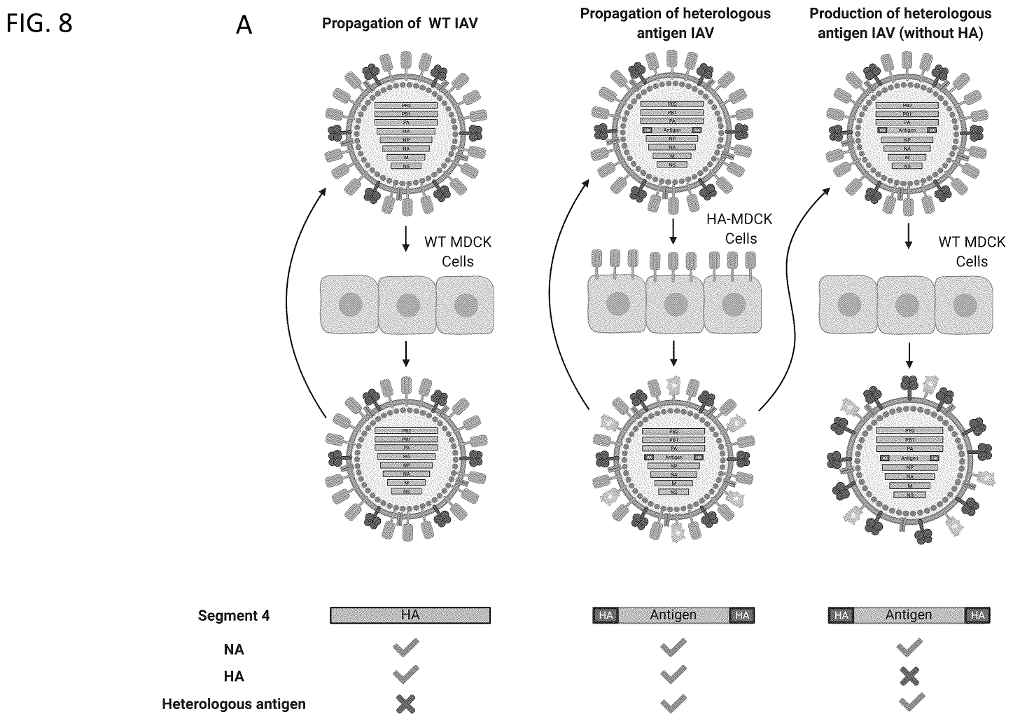

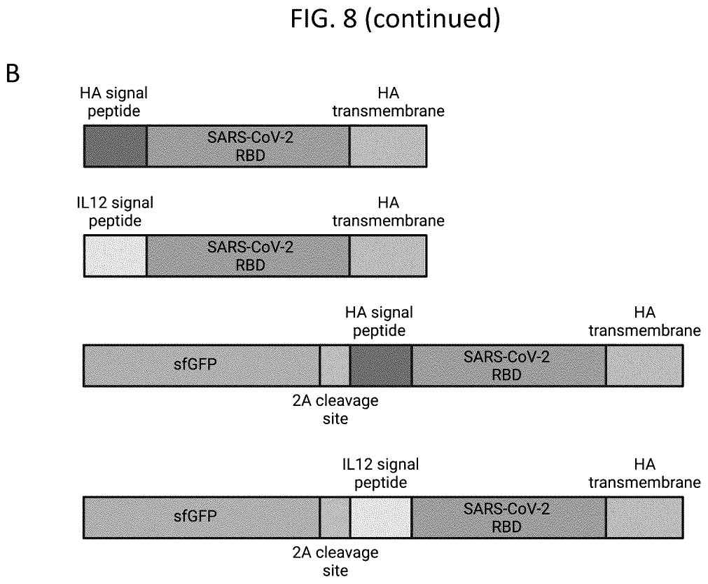

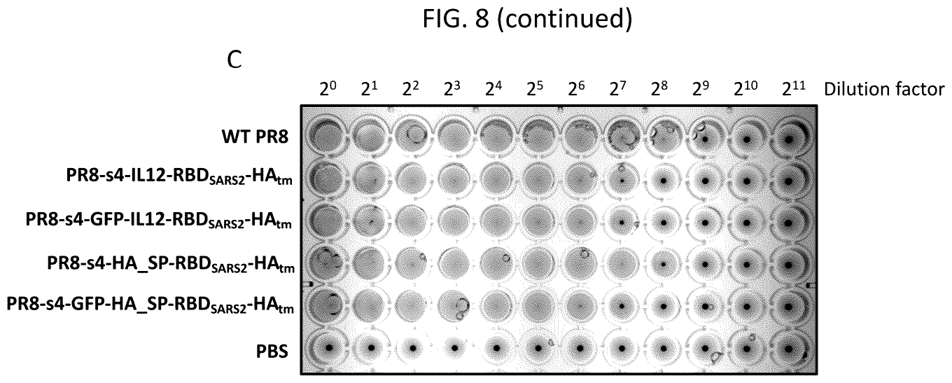

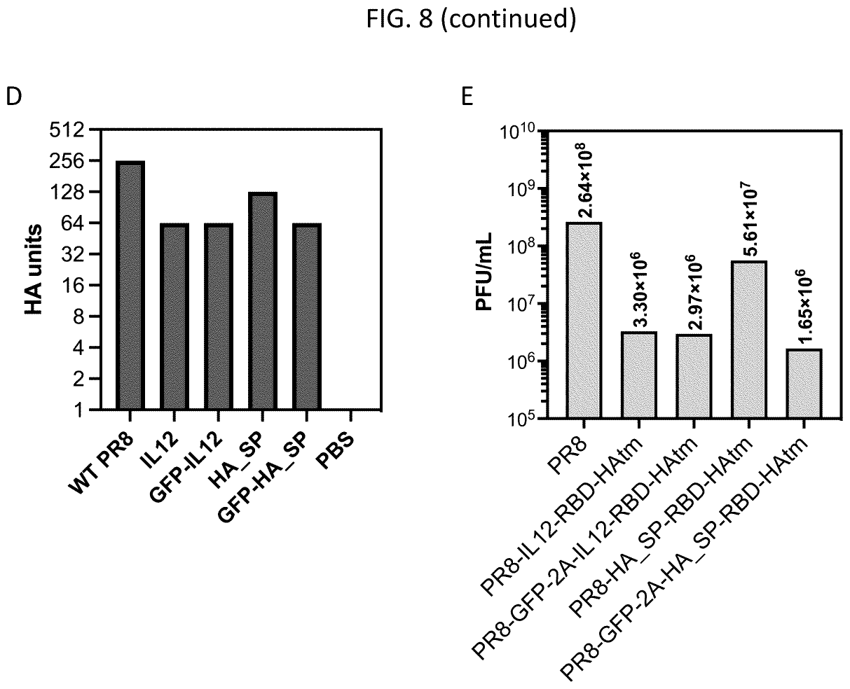

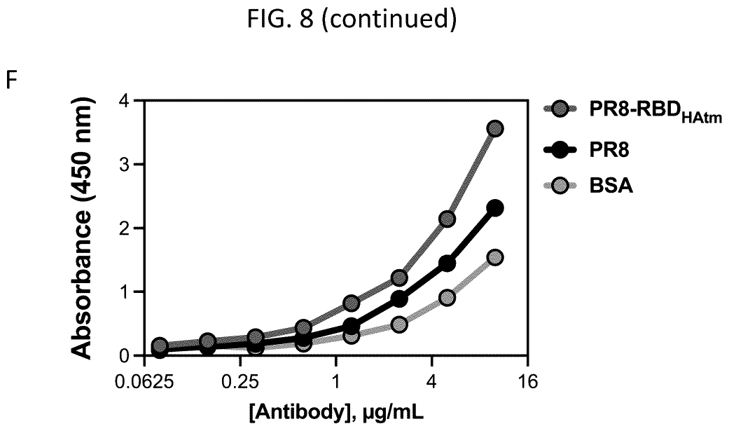

Embodiment 2: In a second embodiment, depicted in FIG. 8, the methods comprise: (a) modifying the HA gene within segment 4 of the genome of an influenza virus to encode a modified HA protein comprising the transmembrane domain of HA and the cytoplasmic tail of HA, thereby rendering the virus replication incompetent; (b) transfecting the modified genome into a first cell line that expresses wild-type HA on its surface; (c) culturing the transfected first cell line to produce viral particles that comprise wild-type HA and the modified segment 4 of step (a); (d) infecting a second cell line that does not express HA with the viral particles produced in step (c); and (e) culturing the infected second cell line to produce replication incompetent viral particles that comprise the modified HA protein.

In Embodiment 2, modification of the HA gene may involve deleting a portion of the HA gene, replacing a portion of the HA gene with exogenous DNA, or introducing a mutation that results in expression of a nonfunctional HA protein. In this embodiment, the modification of the HA gene must retain the transmembrane domain and cytoplasmic tail.

In both Embodiment 1 and Embodiment 2, the first cell line is used to propagate infectious viral particles. The first cell line expresses wild-type HA on its surface, such that the viral particles produced by this cell line comprise wild-type HA and are replication competent. A second cell line that does not express wild-type HA is then used to produce the desired replication incompetent viral particles. In both embodiments, the modified HA protein is expressed on the surface of the final replication incompetent viral particles. However, the difference between these embodiments, is that the modified HA protein is expressed by the second cell line in Embodiment 1, whereas the modified HA protein is expressed from the viral genome in Embodiment 2. In both embodiments, the modified HA protein can further comprise a heterologous protein. However, the heterologous protein is expressed by the second cell line in Embodiment 1, whereas it is expressed from the viral genome in Embodiment 2. Thus, in Embodiment 1, the heterologous protein is shielded from the error-prone mechanisms that are used to replicate the viral genome. As a result, the heterologous protein is less likely to accrue mutations when the viral particles are produced using the methods of Embodiment 1.

In both embodiments, the first cell line may express HA from any suitable nucleic acid construct. Likewise, in Embodiment 1, the second cell line may express the modified HA protein from any suitable nucleic acid construct. For example, the cell lines may express a protein from a plasmid that is transiently transfected into the cell. As used herein, the term “plasmid” refers to a circular double-stranded DNA strand that replicates independently from chromosomal DNA. Alternatively, the cell line may express a protein from a stably integrated gene. Methods of introducing a heterologous gene into the genome of a cell are known in the art and include, without limitation, lentiviral delivery, adeno-associated viral delivery, and CRISPR-based gene editing.

### Methods for Using the Viral Particles:

In a fourth aspect, the present invention provides methods for inducing an immune response in a subject. The methods comprise administering a viral particle or vaccine formulation described herein to the subject.

An “immune response” is the reaction of the body to the presence of a foreign substance (i.e., an antigen). The immune response induced by the present methods may comprise a humoral immune response, a cell-mediated immune response, or both a humoral and cell-mediated immune response. The immune response of a subject to a vaccine may be evaluated indirectly, e.g., through measurement of antibody titers or lymphocyte proliferation assays, or directly, e.g., by monitoring signs and symptoms after challenge with the corresponding pathogen. The protective immunity conferred by the present methods may be evaluated by measuring a reduction in clinical signs, e.g., the mortality, morbidity, temperature, physical condition, or overall health of the subject.

In Example 1, the inventors demonstrate that other proteins besides HA (e.g., NA) were the major drivers of immunity against the heterologous influenza strain H1N1. Thus, in some embodiments, the immune response induced by the method provides protection against a heterologous virus. As used herein, the term “heterologous virus” refers to a virus that is not identical to a reference virus, including both drifted homosubtypic or heterosubtypic viruses.

In preferred embodiments, the methods comprise administering a therapeutically effective amount of the viral particle or vaccine formulation to the subject. As used herein, the term “therapeutically effective amount” refers to an amount of viral particle or vaccine formulation that is sufficient to induce an immune response in a subject receiving the viral particle or vaccine formulation.

In some embodiments, the methods prevent or reduce the symptoms of influenza in the subject. The symptoms of influenza are well-known in the art and include, without limitation, headaches, chest discomfort, cough, sore throat, fever, aches, chills, fatigue, weakness, sneezing, and stuffy nose.

As used herein, the terms “administering” and “administration” refer to any method of providing a pharmaceutical preparation to a subject. Suitable routes of administration include, without limitation, intramuscular, intradermal, intranasal, oral, topical, parenteral, intravenous, subcutaneous, intrathecal, transcutaneous, nasopharyngeal, and transmucosal routes. In some embodiments, the viral particle is administered intramuscularly. The viral particles can be administered as a single dose or in multiple doses. For example, the viral particles may be administered two or more times separated by 4 hours, 6 hours, 8 hours, 12 hours, a day, two days, three days, four days, one week, two weeks, or by three or more weeks. For instance, in Example 1, the viral particles were administered in a prime-boost regime, in which the boost was administer 2-4 weeks after the prime. Thus, in some embodiments, the viral particle is administered to the subject at least twice.

The “subject” to which the present methods are applied may any vertebrate. Suitable vertebrates include, but are not limited to, humans, cows, horses, sheep, pigs, goats, rabbits, dogs, cats, bats, mice, and rats. In certain embodiments, the methods may be performed on lab animals (e.g., mice and rats) for research purposes. In other embodiments, the methods are used to treat commercially important farm animals (e.g., cows, horses, pigs, rabbits, goats, sheep, and chickens) or companion animals (e.g., cats and dogs). In preferred embodiments, the subject is a human.

### Cell Lines:

In a fifth aspect, the present invention provides influenza-susceptible cell lines that express a modified HA protein comprising the transmembrane domain of HA and the cytoplasmic tail of HA. These cell lines can be used to produce replication incompetent viral particles that express the modified HA protein via the methods of Embodiment 1, described above and depicted in FIGS. 1A, 7A, 12A, 15.

As used herein, the term “influenza-susceptible” refers to a cell line that can be infected by influenza. Influenza infects cells by binding to sialic acid-containing receptors present on the cell surface via its HA protein, which triggers viral endocytosis. Thus, an influenza-susceptible cell is a cell that expresses sialic acid on its surface and lacks factors that restrict viral infection (e.g., antiviral proteins).

The cell lines of the present invention are eukaryotic cell lines. Suitable eukaryotic cells include, without limitation, mammalian cells or chicken cells. The cell may be a cell in culture or may be an embryonated chicken egg. Suitable mammalian cells include, without limitation, a MDCK cell, A549 cell, a CHO cell, a HEK293 cell, a HEK293T cell, a HeLa cell, a NS0 cell, a Sp2/0 cell, a COS cell, a BK cell, a NIH3T3 cell, a FRhL-2 cell, a MRC-5 cell, a WI-38 cell, a CEF cell, a CEK cell, a DF-1 cell, or a Vero cell.

In some embodiments, the modified HA protein expressed by the cell further comprises a heterologous protein. Exemplary heterologous proteins are described above in the section titled “Viral particles”. In some embodiments, the heterologous protein is a viral antigen. In some embodiments, the viral antigen is from SARS-CoV-2. In some embodiments, the viral antigen is the receptor binding domain (RBD) of the spike protein from SARS-CoV-2 (SARS-CoV-2 RBD). In specific embodiments, the amino acid sequence encoding the SARS-CoV-2 RBD is SEQ ID NO:11 or a polypeptide having at least 70%, 75%, 80%, 85%, 90%, 95%, or 99% sequence identity to SEQ ID NO:11.

In some embodiments, the modified HA protein further comprises the stalk domain of HA, such that the stalk domain is present on the surface of the viral particle. In specific embodiments, the amino acid sequence encoding the stalk domain is SEQ ID NO:24 or SEQ ID NO:25 or a polypeptide having at least 70%, 75%, 80%, 85%, 90%, 95%, or 99% sequence identity to SEQ ID NO:24 or 25.

The cell lines of the present invention may express the modified HA protein from any suitable nucleic acid construct. For example, the cell line may express a protein from a plasmid that is transiently transfected into the cell (e.g., a plasmid in which the sequence encoding the protein is operably to a promoter that is active in the cell). Alternatively, the cell line may express a protein from a stably integrated gene. Methods of introducing a heterologous gene into the genome of a cell are known in the art and include, without limitation, lentiviral delivery and CRISPR-based gene editing.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1

In the following example, the inventors describe the generation of hemagglutinin (HA)-negative influenza A virus (IAV) particles.

The development of improved and universal anti-influenza vaccines would represent a major advance in the protection of human health. To facilitate the development of such vaccines, understanding how viral proteins can contribute to protection from disease is critical. Much of the previous work to address these questions relied on reductionist systems (i.e., vaccinating with individual proteins or virus-like particles (VLPs) that contain only a few viral proteins). Thus, we have an incomplete understanding of how immunity to different subsets of viral proteins contribute to protection. In the following example, the inventors report the development of a platform in which a single viral protein is deleted from an authentic viral particle that retains the remaining full complement of structural proteins and viral RNA. As a first study with this system, they chose to delete the major influenza A virus (IAV) antigen, the hemagglutinin (HA) protein, to evaluate how the other components of the viral particle contribute en masse to protection from influenza disease. Their results show that, while anti-HA immunity plays a major role in protection from challenge with a vaccine-matched strain, the contributions from other structural proteins were the major drivers of protection against highly antigenically drifted, homosubtypic strains. This work highlights the importance of evaluating the inclusion of non-HA viral proteins in the development of broadly efficacious and long-lasting influenza vaccines.

### Materials and Methods:

### Cell Lines and Viruses

Human embryonic kidney 293T cells (ATCC) were grown in Dulbecco's Modified Eagles Medium (DMEM) supplemented with 5% fetal bovine serum, HEPES, NaHCO₃, GlutaMAX and penicillin-streptomycin. Madin-Darby canine kidney (MDCK) cells (ATCC) were cultured in minimal essential medium (MEM) supplemented with 5% fetal bovine serum, HEPES, NaHCO₃, GlutaMAX and penicillin-streptomycin. The PR8 HA protein was introduced into a MDCK cell line via lentivirus delivery; cells were then grown under the same conditions as unmodified MDCK cells. All cell lines were grown at 37° C. in 5% CO₂. The influenza A virus challenge strains A/Puerto Rico/8/1934 (PR8) and A/California/04/2009 (Cal09) were propagated in embryonated chicken eggs. As viral strains may acquire mutations during laboratory passaging, the strains used for challenge in this study were sequenced via sanger sequencing. GenBank accession numbers for the A/Puerto Rico/8/1934 viral genes (with deviations noted) are: PB2, AF389115.1; PB1, CY148249.1 (G1557T, silent; C1773T, silent); PA, AF389117.1; HA, AF389118.1 (A651C, I207L); NP, AF389119.1; NA, AF389120.1; MP, AF389121.1; NS, AF389122.1. GenBank accession numbers for the A/California/04/2009 (Cal09) viral genes (with deviations noted) are: PB2, MN371615.1; PB1, MN371613.1 (G498A, silent); PA, MN371611.1 (G2022A, Q670H); HA, MN371616.1 (C655A, A212E; A739G, Q240R; G1395A, V459M; T1487C, silent); NP, MN371617.1 (A335G, D101G); NA, MN371610.1; MP, MN371612.1; NS, MN371614.1.

### Experimental System for Producing HA-Negative Viral Particles

To generate HAtm-GFP MDCK cells, the HA transmembrane and cytoplasmic domains were fused to GFP. The gene fragment was synthesized (IDT) and cloned into lentivirus vector pLEX. Lentiviruses were packaged on 293T cells and used to transduce MDCK cells. The resultant transduced cell line was passaged in the presence of puromycin and maintained at low passage numbers to produce HA-negative viruses. Segment 4 mCherry IAV was designed and rescued based on the bicistronic pDZ rescue plasmid system. Viral sequences were based on the reverse-genetic rescue plasmids from the PR8 H1N1 background as previously described (40, 41). To remove the HA protein ORF from segment 4, the middle of the segment was deleted and only the 5′ terminal 99 nt and the 3′ terminal 150 nt (based on the nucleotide positions in the positive sense RNA) were preserved to serve as packaging signals. Further, to prevent inappropriate early translation, all ATGs in the 3′ packaging signal were mutated to TTG. A consensus Kozak sequence and the mCherry gene (flanked by 3′ EcoRV and 5′ PmeI restriction sites) were inserted in between the packaging signals to generate the final segment. Plasmids corresponding to seven WT PR8 viral segment along with segment 4 mCherry and pLEX-HA plasmid were transfected into 293T cells using TransIT-LT1 (Mirus). The rescued viruses lacking the HA gene were then propagated and tittered on HA-MDCK cells.

### Propagation of Viral Stocks and Vaccine Formulation

MDCK cells were infected at an MOI of 0.01 with wild-type PR8 to grow WT IAV. HAtm-GFP were infected at an MOI of 5 with segment 4 mCherry IAV to produce HA-negative IAV. Virus supernatants were layered on top 30% sucrose/PBS and were ultra-centrifuged for 1 h at 27,500 rpm for concentration. For vaccine formulation, the concentrated viral particles were assayed for neuraminidase activity and then normalized. The protein concentrations of the normalized preparations were then quantified by Bradford assay and 10 μg of the HA-negative viral preparation was administered in a given vaccination. The total amount of the WT control vaccine was allowed to fluctuate to match the amount of the NA in the HA-negative preparation and usually slightly less total protein was used for WT vaccination. Viral preparations were inactivated by incubating with 0.02% formalin for 30 min and then dialyzed by Slide-A-Lyzer cassettes (Thermo Scientific).

### Western Blotting

Equal amounts of protein were loaded into 4-20% acrylamide gels (Bio-Rad) and transferred to nitrocellulose membrane. PBS with 5% (w/v) non-fat dried milk and 0.1% Tween-20 was used for blocking for 2 h at 4° C. Primary antibodies were then incubated with the membrane overnight at 4° C. Antibodies used were mouse anti-HA (PY102), mouse anti-NA (4A5 (30)), mouse anti-M (M2E10) and mouse anti-GFP (Cell Signaling Technology, 2955S). Membranes were washed five times in PBS with 0.1% Tween-20 and then anti-mouse-HRP or anti-rabbit-HRP secondary antibodies (GE Healthcare) were incubated with the blots for 1 h. The membrane was then washed five times and Clarity or Clarity Max ECL substrate (Bio-Rad) was added before being exposed to film and developed. For densitometry analysis, quantification was performed with ImageJ (NIH) and values were normalized prior to statistical analysis.

### Vaccination and Animal Challenge

Six- to ten-week old C57BL/6 female mice were used for all experiments. For vaccination, the vaccine was administered intramuscularly at one injection site. After 2 to 4 weeks, mice were boosted in the same fashion and given another 2 to 3 weeks before challenge or the collection of serum. For infection, mice were administered 40 μL of the virus (10,000 PFU for PR8, 24,000 PFU for Cal09) intranasally after anesthesia with a ketamine-xylazine mixture. Mice were weighed daily and euthanized once their body weight reached <80% of the starting weight as a humane endpoint. Euthanasia was performed via CO₂ as the primary method and a bilateral thoracotomy was performed as the secondary method.

### ELISA

For whole virus ELISAs, virions were concentrated using a 30% sucrose cushion for 1 h at 25,700 rpm on the Sorvall TH-641 swinging bucket rotor and then resuspended in PBS. For HA and NA ELISAs, PR8 HA protein was expressed by 293T cells and purified with immobilized metal affinity chromatography. PR8 NA, Cal09 HA and NA were obtained through BEI Resources (NR-19235, NR-51668, NR-19234). 96-well plates were coated at 4° C. with protein using a carbonate buffer overnight. For the PR8 M2 cell-based ELISA, the pLEX-M2 plasmid was transfected into 293T cells in suspension by TransIT-LT1 (Mirus), then cells were seeded into ninety-six-well plates and grown for 48 h. Cells were fixed with 4% paraformaldehyde (PFA)/PBS before addition of the serum. Serum samples were then diluted and added to the wells. Bound Ab was detected by using sheep anti-mouse HRP-conjugated antibody (GE Healthcare). Color was developed by using tetra-methyl-benzidine (TMB) substrate (Thermo Scientific), and reactions were stopped with 2M sulfuric acid. Absorbance was measured at 450 nm on a plate reader. Area under the curve (total area) was calculated with Prism (Graphpad) software using the average of the blank samples as the background cutoff.

### HAI Assay

Sera was treated with receptor-destroying enzyme (RDE, Denka Seiken) at a 1:4 dilution at 37° C. for 20 h followed by inactivation at 56° C. for 30 min and further dilution to 1:10 with PBS. Sera was 2-fold serially diluted in v-bottom microtiter plates. Virus adjusted to 4 HA units in 25 μL was added to each well. The plates were incubated at room temperature for 15 min followed by the addition of 50 μL of 0.5% chicken (for PR8) or turkey (for Cal09) erythrocytes (Lampire Biologicals) in PBS. The reaction mixture was then allowed to settle for 30 min at room temperature. Wells were examined visually for inhibition of HA. HAI titers were the reciprocal of the highest dilution of serum that completely prevented HA.

### Plaque Assay

Viral titers in lungs were determined using a standard plaque assay protocol on MDCK cells. Virus was serially diluted, and after incubation with the cells for 1 h at 37° C., virus was removed and a 1% agar overlay containing TPCK-trypsin was applied. After incubation at 37° C. for 48 to 72 h, assays were fixed in 4% PFA for 3 h. Serum from WT PR8- or WT Cal09-infected mice was diluted in antibody dilution buffer (5% (w/v) non-fat dried milk and 0.05% Tween-20 in PBS) and incubated on cells at 4° C. for 12 h. Cells were then washed and incubated for 2 h in diluted sheep anti-mouse HRP-conjugated antibody in antibody dilution buffer. Assays were then washed with PBS and exposed to 0.5 ml of True Blue peroxidase substrate (KPL) for 15 min. Plates were then washed with water and dried before the plaques were counted.

### Microscopy

Fluorescent images were taken using HA-MDCK cells infected with 0.1 MOI segment 4 mCherry IAV or HAtm-GFP MDCK cells infected with 10 MOI segment 4 mCherry IAV. At 48 h post infection, cells were incubated with Hoechst 33342 stain (Life Technologies) to allow for the staining of nuclei, and imaging was performed on the Zoe fluorescent cell imager (Bio-Rad). For H&E stained slides, mouse lungs tissues were fixed in 4% PFA/PBS at 4° C. for more than 16 h. Samples were embedded in paraffin and sectioned after dehydration and wax immersion, slides were then rehydrated and stained with H&E as per standard protocols. Microscopy was done on a Zeiss Axio Imager microscope. Images were then processed with ImageJ (NIH).

### Statistical Analysis

Data were analyzed using Prism software (GraphPad). Values below the limit of detection were assigned a value of one half of the LOD (LOD/2) in subsequent analyses. Unless otherwise noted, significance was determined by using a Students T-test or a one-way analysis of variance (ANOVA) followed by Tukey's post-hoc analysis. Bodyweight changes and survival after viral challenge were analyzed by a two-way ANOVA followed by a Sidak's multiple comparisons test or a log-rank (Mantel-Cox) test, respectively. Asterisks in all figures indicated significance as follows: *P<0.05; **P<0.001.

### Results:

### Design and Generation of “Authentic” Viral Particles Lacking the HA Protein

Our initial goal was to generate matched, inactivated viral particle-based vaccines that only differ from naturally occurring viral particles by their lack of the HA protein. To accomplish this goal, we first took advantage of a well-established approach to delete the HA protein from segment 4 of the viral genome and replace it with an irrelevant protein (in this case mCherry) flanked by the segment 4 packaging signals (25). Using the reverse genetics system, plasmids corresponding to seven WT viral segments from A/Puerto Rico/8/1934 (PR8) and the segment 4 mCherry vector (along with an HA protein expression plasmid) were transfected into 293T cells. The resultant viral particles were subsequently propagated on MDCK cells stably expressing the PR8 HA protein (FIG. 1A). These viral particles, despite not encoding a gene for HA still harbor cell-expressed HA on the viral particle and efficiently propagate themselves when passaged on the HA-expressing cell line; we refer to this virus as the Segment 4 mCherry IAV (FIG. 1B).

It has been reported that IAV budding is inefficient in the absence of the HA protein (26, 27). Therefore, in order to produce viral particles that efficiently bud without an HA protein, we generated a second MDCK cell line that expressed the transmembrane and cytoplasmic domains of HA fused to GFP in place of the normal HA ectodomain (SEQ ID NO:1; FIG. 1A). With this cell line, we were able to use the segment 4 mCherry IAV virus to infect the HAtm-GFP MDCK cell line to produce viral particles that replace the HA-ectodomain with GFP (FIG. 1A,C). After collection and concentration of the HA-negative virus particles (along with a corresponding WT virus propagated on MDCK cells), virion protein composition was analyzed via western blotting. After normalization to the viral matrix (M1) protein, we evaluated HA levels. As expected, HA was detected in the WT viral particles but absent in the HA-negative IAV prep. However, blots for GFP showed the reciprocal trend (FIG. 1D). Interestingly, slightly more NA and moderately more M2 were present on the HA-negative viral particle, suggesting that virus lacking the full-length HA (but still packaging an HA “replacement” protein) allows for increased abundance of the other surface exposed structural proteins in viral particles (FIG. 1D-F).

Vaccination with WT and HA-Negative IAV Particles Leads to Differential Immune Responses and Protection Against Homologous Viral Challenge

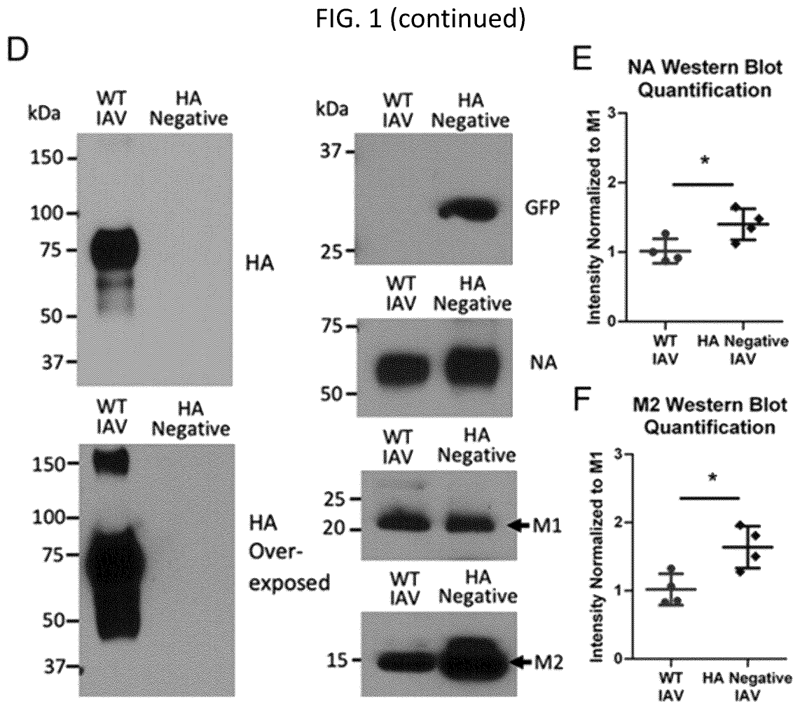

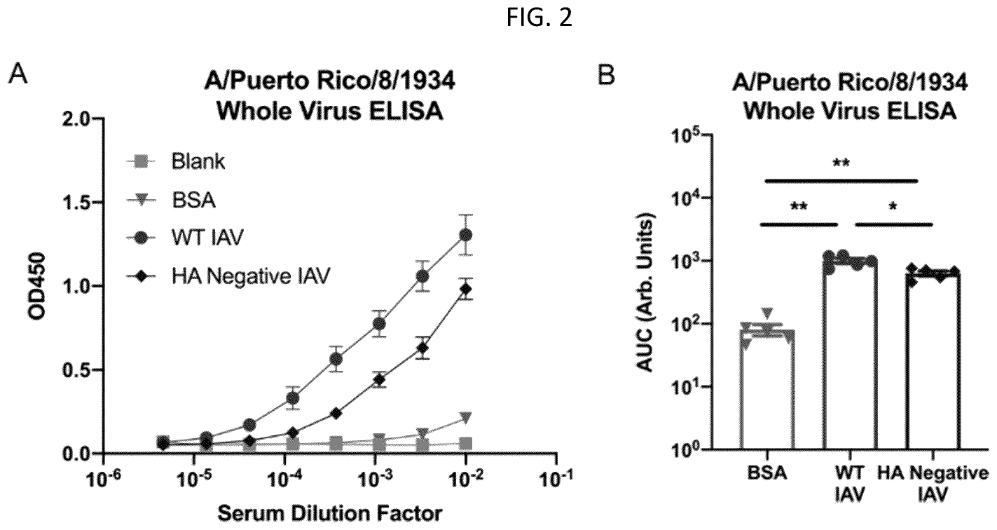

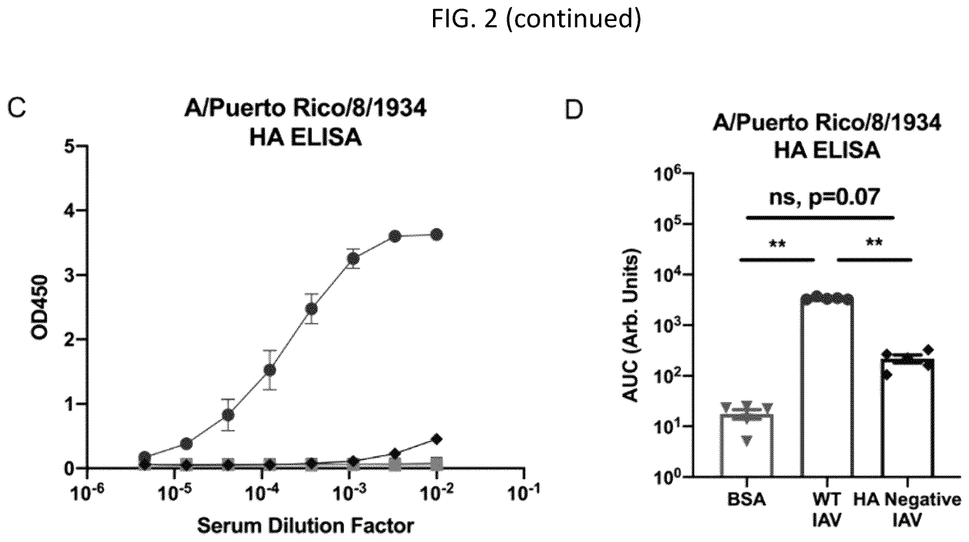

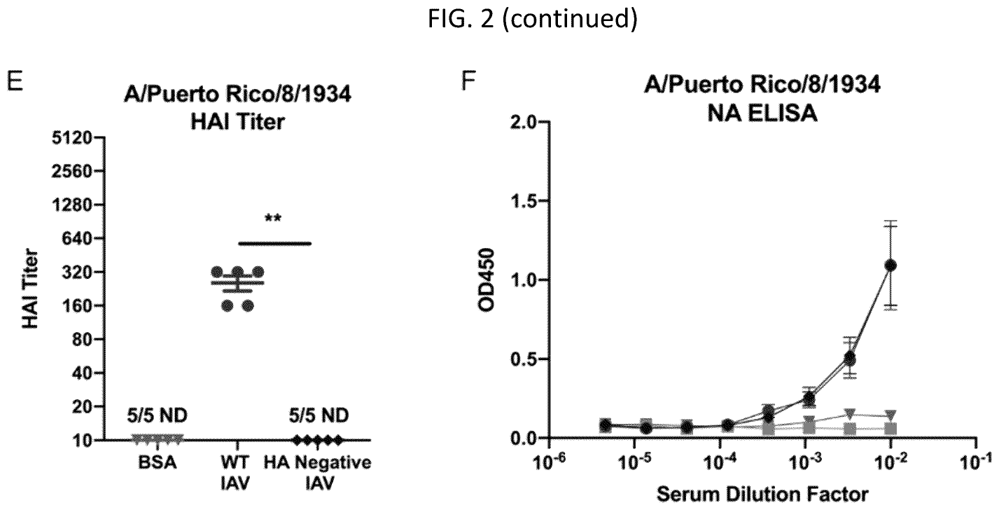

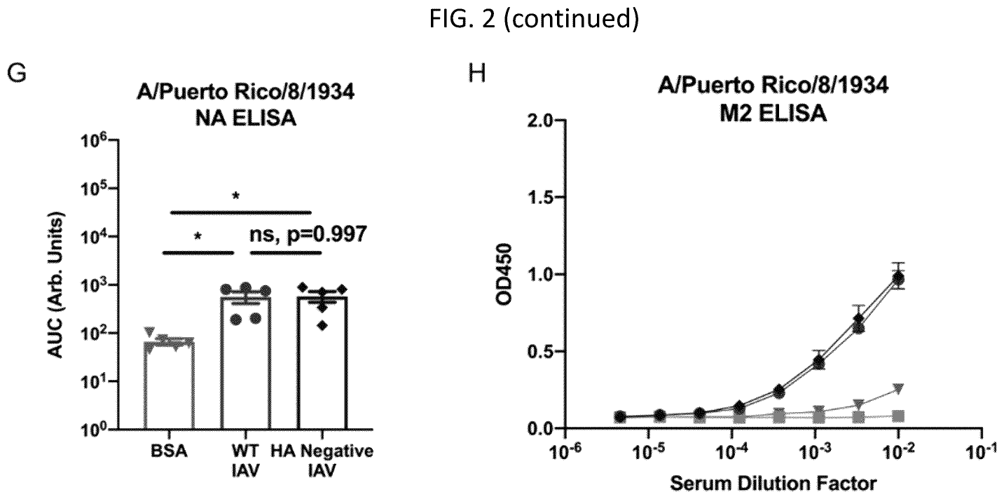

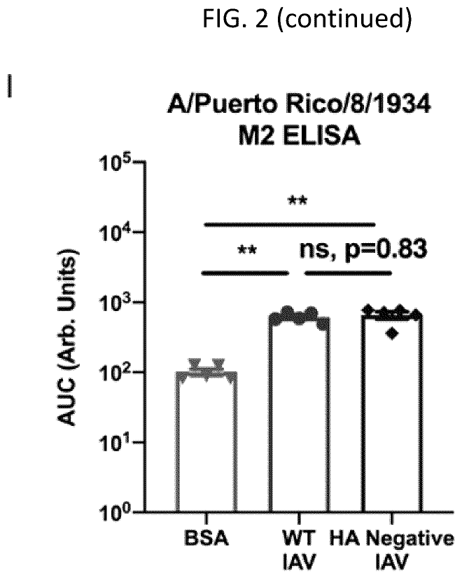

We next evaluated the immune responses generated after vaccination with either the WT or HA-negative viruses. Because HA-negative virus particles are not “infectious” due to the lack of this receptor binding protein, we tested the viral particles in the context of inactivated vaccine formulations. C57BL/6 mice received an intramuscular prime and single boost with formalin-inactivated viral preparations that were normalized via NA content, or BSA as a control. After the boost, immune sera were collected and reactivity to the parental PR8 strain was evaluated via ELISA. We chose to focus on serum IgG antibodies as our experimental readout as mucosal antibodies and CD8 T-cell responses are limited after inactivated influenza vaccination (28, 29). While both virus preps were immunogenic, the overall reactivity to the WT-virus derived vaccine was higher relative to the HA-negative virus (FIG. 2A,B). This difference was due primarily to high levels of anti-HA antibodies uniquely present in the WT vaccine group (FIG. 2C,D). Further, as expected, serum from the WT vaccinated animals had high hemagglutination inhibition (HAI) activity, while the serum from the HA-negative vaccine group had none (FIG. 2E).

We also tested for serum reactivity against the NA and M2 viral proteins. Although reactivity was above background levels, we were unable to detect a difference in reactivity against either antigen between the two groups (FIG. 2F-I). Thus, it appears that, in the absence of HA and under these vaccination conditions, serum IgG responses against the other viral proteins are not appreciably boosted. The inherent immunogenicity and immune responses against all of the non-HA structural proteins together, however, are not insignificant and are well above the BSA vaccine group (FIG. 2A,B).

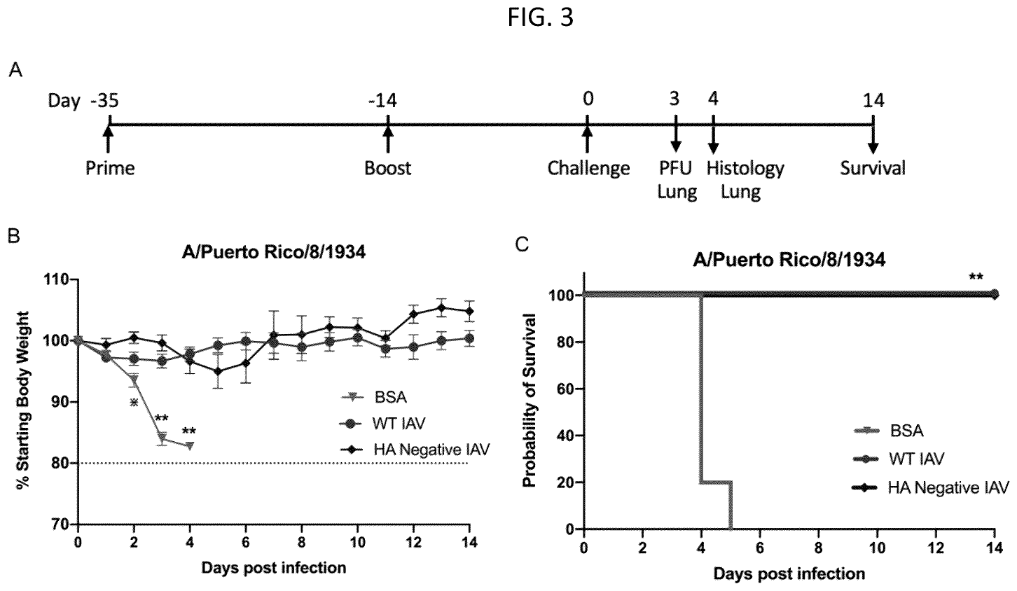

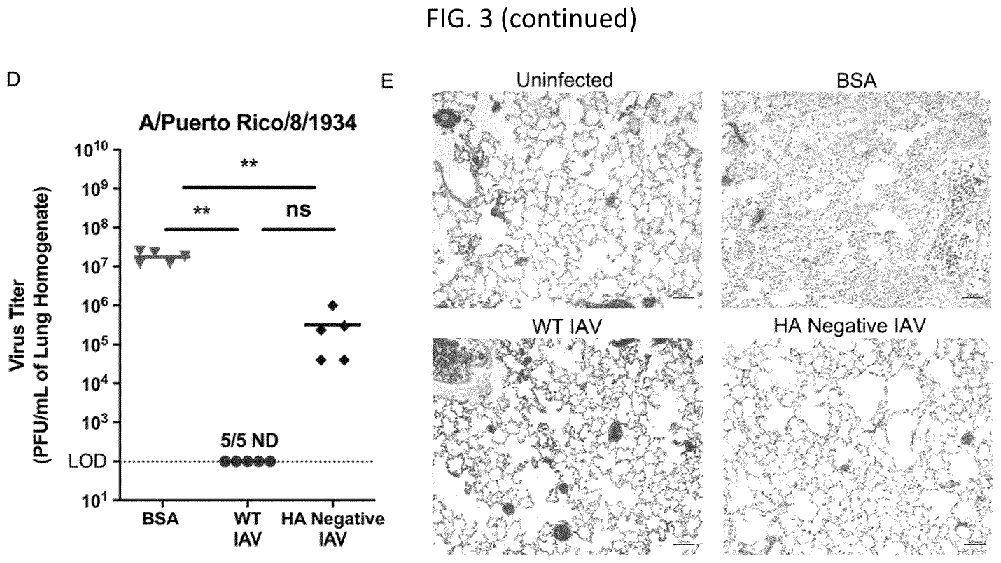

We next investigated how the different immune profiles raised against these two vaccines would affect protection from homologous viral challenge. Mice again received a prime and a single boost of the PR8 based vaccine, followed by a lethal PR8 challenge (FIG. 3A). As a primary readout of protection from challenge, bodyweight and mortality were monitored for 14 days post-challenge. The control BSA group rapidly lost weight and reached the humane endpoint within five days of infection, however all of the vaccinated mice ultimately survived the challenge (FIG. 3B,C). While the survival between the two viral vaccine groups was the same, there was reproducible dip in bodyweight after infection of the HA-negative vaccine group (FIG. 3B).

To understand how the different vaccines were affecting viral burden, we repeated the vaccination and challenge experiment and harvested lungs at 3 days post infection. In contrast to the bodyweight and survival, there was a striking difference in this metric of vaccine protection; while the HA-negative vaccine significantly reduced viral titers (by -2 orders of magnitude) compared to the control, the WT vaccine was much more effective and reduced viral titer by at least 5 orders of magnitude to below our limit of detection (FIG. 3D). Despite the differences in viral titer, lung histopathologic analysis at four days post-infection failed to reveal obvious differences between the WT IAV and HA-negative IAV groups (FIG. 3E). Together, these data demonstrate that while HA-negative IAV vaccines can provide significant protection against influenza disease, immunity against the HA protein contributes substantially to reducing viral titers.

Non-Hemagglutinin Structural Proteins can be Major Drivers of Protection from Highly Drifted, Homosubtypic Strains

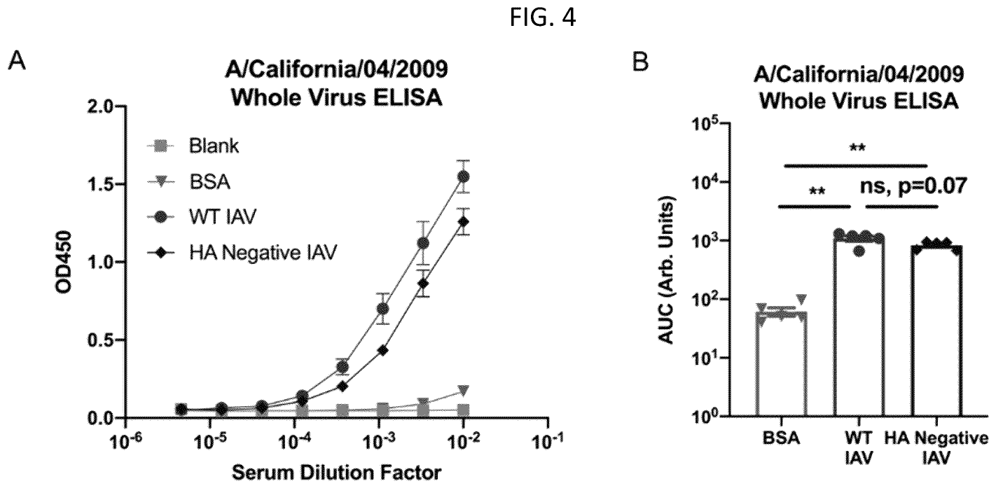

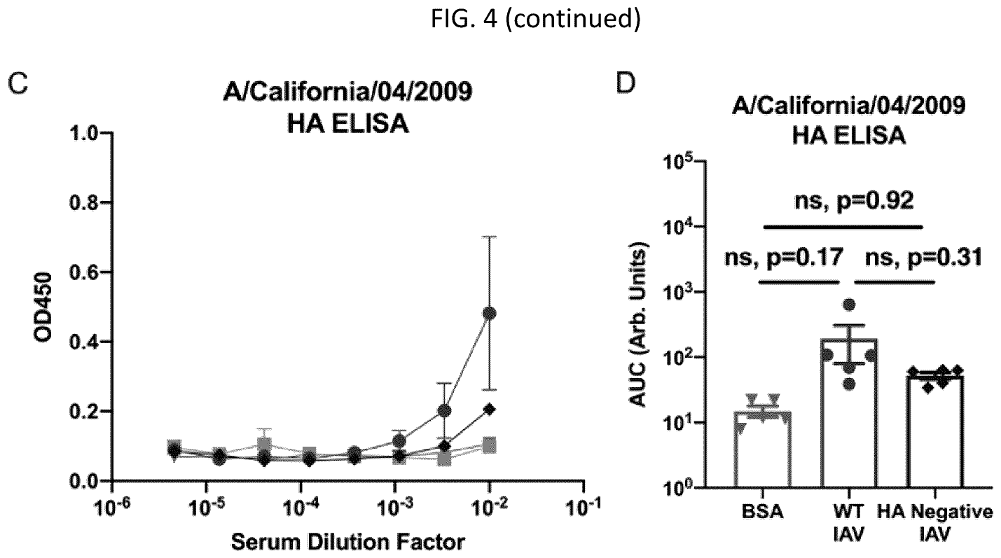

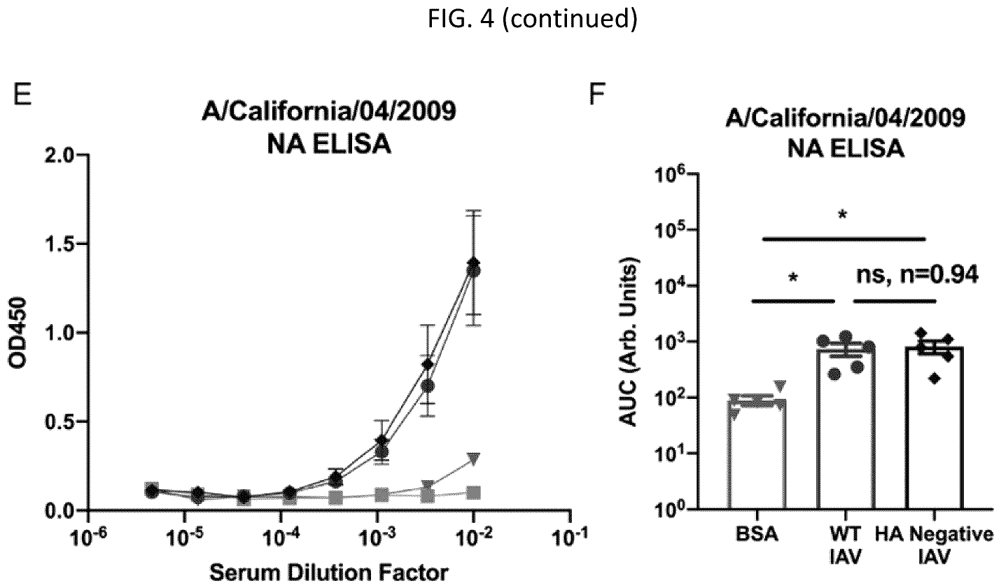

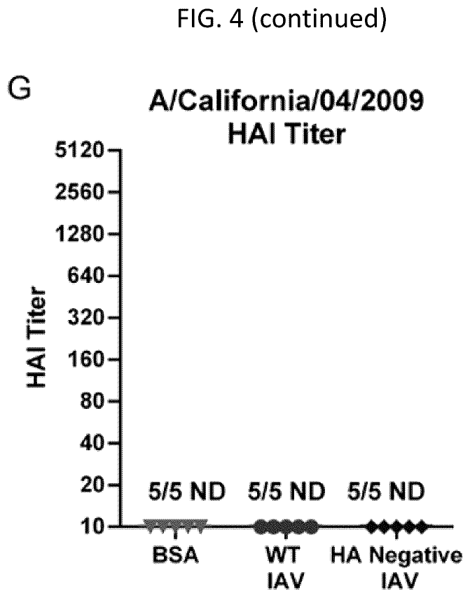

Due to the relatively higher conservation of many of the non-HA structural proteins, we were interested in defining how the immunity induced by our HA-negative vaccine would compare to WT vaccines in the context of an antigenically distinct H1N1 virus. We therefore repeated our PR8-based vaccination scheme, as described in FIG. 2, and assayed for reactivity against the A/California/04/09 (Cal09) virus. This is a prototype 2009 “swine” pandemic strain, and while still an H1N1, it is highly divergent from PR8 which was isolated in 1934. ELISAs against the intact Cal09 viral particle, as well as the HA and NA proteins individually, revealed similar trends to the PR8 ELISAs in FIG. 2, although the magnitudes of the changes were much smaller (FIG. 4A-F). While total reactivity against the intact Cal09 viral particle and the HA protein trended higher in the WT vaccine group compared to the HA-negative group, these differences were not reproducibly statistically significant (FIG. 4A-D). Further, the magnitude of HA reactivity was in fact quite low and close to background for all groups (FIG. 4C,D). Cal09 NA reactivity was higher than HA reactivity, and identical between the WT and HA-negative vaccine groups (FIG. 4E,F). As expected, none of the sera from any of the vaccinated groups revealed any detectable HAI activity against Cal09 (FIG. 4G).

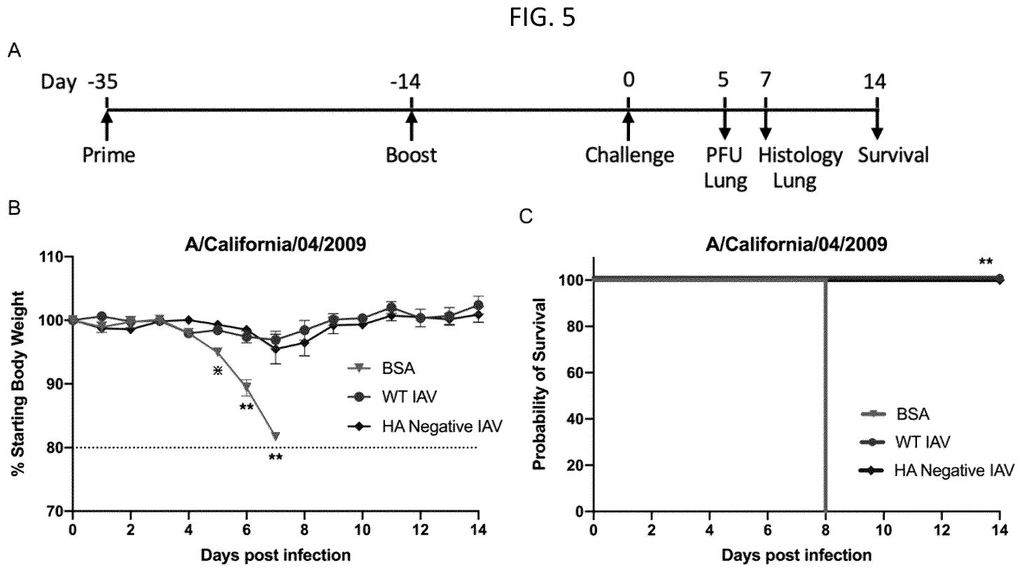

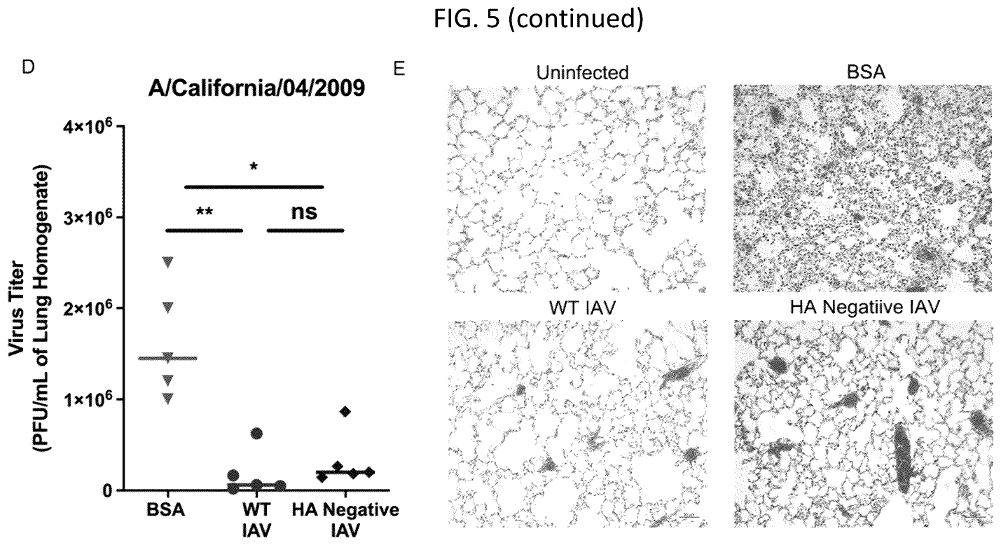

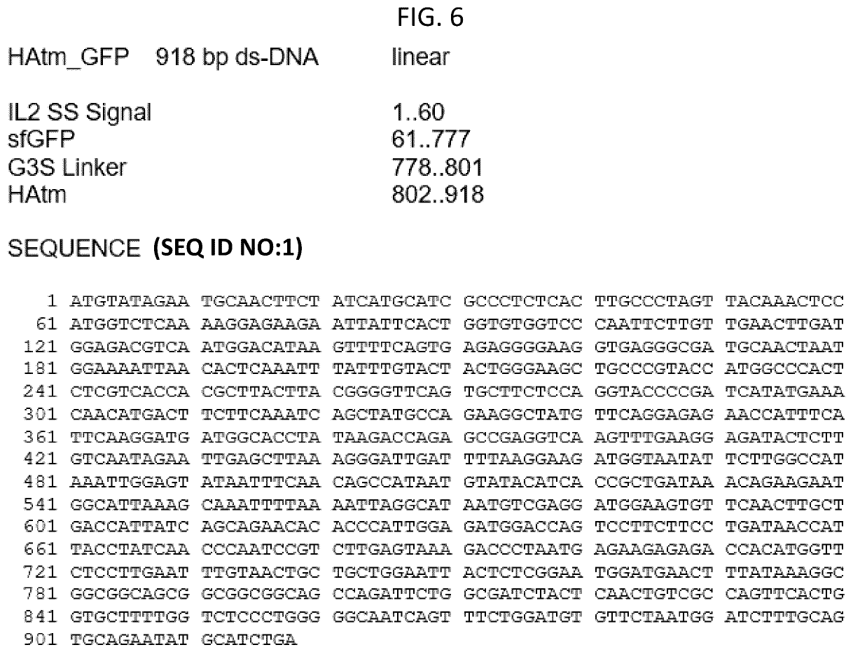

We next turned our attention to defining the protection that PR8-based HA-negative and WT IAV vaccines would provide against Cal09 challenge. Mice again received an inactivated prime and a single boost, followed by a lethal infection with the Cal09 virus (FIG. 5A). As expected, all of control BSA vaccinated mice succumbed to infection. At this infectious dose of Cal09, all the mice from both viral vaccine groups survived challenge, but in contrast to the PR8 challenge, no obvious differences in bodyweight loss between the two groups were observed (FIG. 5B,C). Since the largest differences during the homologous challenge experiments were in the infectious viral titers from the lungs, we also analyzed lung titer from the Cal09 challenge experiments. While both vaccine groups had significantly less virus in the lungs compared to control, there was no difference between the two viral vaccine groups (FIG. 5D). H&E stained lung sections showed significantly less immune cell infiltration in both viral vaccine groups compared to control, but as expected from the viral titer data, we failed to observe any striking differences between the vaccine groups (FIG. 5E). Together, these data support the notion that as viral strains diverge, the non-HA structural proteins can play critical and efficacious roles in vaccine mediated protection.

### Discussion

The development of improved influenza virus vaccines is of the highest importance to protect public health. To facilitate that goal, a better understanding of how different viral proteins contribute to immunity against both matched and antigenically drifted strains is needed. In this study, we generated “authentic” influenza virus particles deficient for the HA protein to understand how all the other structural proteins, together, contribute to vaccine efficacy. We show that while anti-HA immunity plays a major role in reducing viral titers during homologous challenge, the contributions of HA become less important when evaluating protection from disease and even more so against highly drifted strains. In fact, we failed to detect any significant differences between WT and HA-negative vaccines in reducing viral titer or in mediating protection from a highly drifted, homosubtypic challenge.

These data highlight that, particularly with respect to universal influenza vaccine development, the “other”, non-HA structural proteins should be given serious attention. In most seasonal influenza vaccines, the formulation is based exclusively on HA content and the content of at least some of the other viral proteins have been shown be highly variable (30). Our data suggest that while this is unlikely to affect protection against matched strains (and indeed, recombinant HA only vaccines (31) are efficacious and FDA approved), ignoring the non-HA viral proteins could have significant implications for the duration or breadth of protection. With that said, it is also important to avoid over-generalization of the results of our study. We only generated a HA-negative vaccine in the PR8 viral background and evaluated immunity against one other strain, Cal09. At a minimum, our data shows the magnitude of the protective effect that the non-HA structural proteins can have mediating protection from drifted strains. However, it is also worth highlighting that our data are consistent with previous reports of “single cycle” live attenuated viral vaccines, which although they are genetically deleted for the HA protein, display HA on the incoming viral particle and perform one round of replication after vaccination (32-34). This approach elicits little HA-directed immunity but is also associated with strong protection from viral challenge, in general agreement with our conclusions.

One major question that was not answered by our study was the definition of the relative contributions of each of the non-HA proteins to the protection phenotype. We believe that it is likely that anti-NA antibodies are major contributors, as vaccination with the NA protein alone has been reported to be sufficient to mediate protection from influenza disease (30, 35, 36). Further, although we focused our analysis on serum antibodies against the surface exposed viral proteins, T cell responses against proteins like NP and M1 (37) may be modulated in the absence of the immunodominant HA protein and could have contributed to the observed protection. However, future studies will be required to resolve these questions.

It is also important to note that our HA-negative vaccine preparation was not truly “matched” to the WT comparison with respect to non-HA proteins. We know that at a minimum, NA and M2 content in the HA-negative viral particle was altered. Although we normalized our vaccine formulation by NA activity and could not subsequently detect a difference in reactivity to these proteins in our assays, altered immune responses (antibody-mediated or otherwise) to these or other structural proteins may have contributed to the protective effects we observed during challenge. Additionally, our HA-negative IAV particles packaged GFP in place of HA. While we consider GFP an “irrelevant” protein with respect to influenza virus protective immunity, GFP itself may have some immunogenic properties (38) which should also be evaluated in the future.

In addition to its use as a tool to probe vaccine-mediated immunity in the absence of HA, our approach also has the potential to serve as a modular platform with which to package proteins onto an authentic viral particle. We have successfully packaged GFP, which in no meaningful way contributed to viral biology. Although not tested in this study, it is likely that we can fuse a range of proteins to the transmembrane domain and cytoplasmic tail of HA and use this approach to efficiently incorporate them into the viral particle. In contrast to genetically encoding them in a fully replication competent virus, encoding the “foreign” proteins in the cell line negates the viral mechanisms to mutate or eliminate the protein. This would likely lead to high stability of the foreign protein on the viral particle. Finally, it has also been shown that anti-HA antibodies can interfere with neuraminidase activity via steric interference (39). Our HA-negative viral particles may be an attractive reagent to probe the effects of anti-NA antibodies without concerns of interference from HA antibodies.

In conclusion, many reports have evaluated the relative contributions of different IAV proteins to immunity when administered alone or in limited combinations. To our knowledge, no previous study has generated “complete” viral particles that only lack the HA protein. Using a combination of viral genetic manipulations and helper cell lines, we were able to produce inactivated IAV vaccines that allowed us to disentangle the antigenic contributions of HA from that of all the other viral structural proteins. Our results suggest that, especially for vaccines designed to provide broad or long-lasting protection, the non-HA structural proteins should be carefully evaluated and may be important components of next-generation anti-influenza vaccines.

### REFERENCES

1. Troeger C E, Blacker B F, Khalil I A, Zimsen S R M, Albertson S B, Abate D, Abdela J, Adhikari T B, Aghayan S A, Agrawal S, Ahmadi A, Aichour A N, Aichour I, Aichour M T E, Al-Eyadhy A, Al-Raddadi R M, Alahdab F, Alene K A, Aljunid S M, Alvis-Guzman N, Anber N H, Anjomshoa M, Antonio C A T, Aremu O, Atalay H T, Atique S, Attia E F, Avokpaho EFGA, Awasthi A, Babazadeh A, Badali H, Badawi A, Banoub J A M, Barac A, Bassat Q, Bedi N, Belachew A B, Bennett D A, Bhattacharyya K, Bhutta Z A, Bijani A, Carvalho F, Castaneda-Orjuela C A, Christopher D J, Dandona L, Dandona R, Dang A K, Daryani A, Degefa M G, Demeke F M, et al. 2019. Mortality, morbidity, and hospitalisations due to influenza lower respiratory tract infections, 2017: an analysis for the Global Burden of Disease Study 2017. Lancet Respiratory Medicine 7:69-89.
2. Paget J, Spreeuwenberg P, Charu V, Taylor R J, Iuliano A D, Bresee J, Simonsen L, Viboud C, Global Seasonal Influenza-associated Mortality Collaborator N, Teams* GLC. 2019. Global mortality associated with seasonal influenza epidemics: New burden estimates and predictors from the GLaMOR Project. J Glob Health 9:020421.
3. Rolfes M A, Foppa I M, Garg S, Flannery B, Brammer L, Singleton J A, Burns E, Jernigan D, Olsen S J, Bresee J, Reed C. 2018. Annual estimates of the burden of seasonal influenza in the United States: A tool for strengthening influenza surveillance and preparedness. Influenza Other Respir Viruses 12:132-137.
4. Principi N, Camilloni B, Alunno A, Polinori I, Argentiero A, Esposito S. 2019. Drugs for Influenza Treatment: Is There Significant News? Frontiers in Medicine 6.
5. Wei C J, Crank M C, Shiver J, Graham B S, Mascola J R, Nabel G J. 2020. Next-generation influenza vaccines: opportunities and challenges. Nat Rev Drug Discov 19:239-252.
6. Houser K, Subbarao K. 2015. Influenza vaccines: challenges and solutions. Cell Host Microbe 17:295-300.
7. Noah D L, Hill H, Hines D, White E L, Wolff M C. 2009. Qualification of the hemagglutination inhibition assay in support of pandemic influenza vaccine licensure. Clin Vaccine Immunol 16:558-66.
8. Yu X, Tsibane T, McGraw P A, House F S, Keefer C J, Hicar M D, Tumpey T M, Pappas C, Perrone L A, Martinez O, Stevens J, Wilson I A, Aguilar P V, Altschuler E L, Basler C F, Crowe J E, Jr. 2008. Neutralizing antibodies derived from the B cells of 1918 influenza pandemic survivors. Nature 455:532-6.
9. Neu K E, Dunand C J H, Wilson P C. 2016. Heads, stalks and everything else: how can antibodies eradicate influenza as a human disease? Current Opinion in Immunology 42:48-55.
10. Krammer F, Palese P. 2015. Advances in the development of influenza virus vaccines. Nat Rev Drug Discov 14:167-82.
11. Rajao D S, Perez D R. 2018. Universal Vaccines and Vaccine Platforms to Protect against Influenza Viruses in Humans and Agriculture. Front Microbiol 9:123.
12. Sui J, Hwang W C, Perez S, Wei G, Aird D, Chen L M, Santelli E, Stec B, Cadwell G, Ali M, Wan H, Murakami A, Yammanuru A, Han T, Cox N J, Bankston L A, Donis R O, Liddington R C, Marasco W A. 2009. Structural and functional bases for broad-spectrum neutralization of avian and human influenza A viruses. Nat Struct Mol Biol 16:265-73.
13. Ekiert D C, Bhabha G, Elsliger M A, Friesen R H, Jongeneelen M, Throsby M, Goudsmit J, Wilson I A. 2009. Antibody recognition of a highly conserved influenza virus epitope. Science 324:246-51.
14. Nachbagauer R, Krammer F. 2017. Universal influenza virus vaccines and therapeutic antibodies. Clin Microbiol Infect 23:222-228.
15. Eichelberger M C, Wan H Q. 2015. Influenza Neuraminidase as a Vaccine Antigen. Influenza Pathogenesis and Control—Vol Ii 386:275-299.
16. Kilbourne E D, Johansson B E, Grajower B. 1990. Independent and disparate evolution in nature of influenza A virus hemagglutinin and neuraminidase glycoproteins. Proc Natl Acad Sci USA 87:786-90.
17. Couch R B, Atmar R L, Franco L M, Quarles J M, Wells J, Arden N, Nino D, Belmont J W. 2013. Antibody correlates and predictors of immunity to naturally occurring influenza in humans and the importance of antibody to the neuraminidase. J Infect Dis 207:974-81.
18. Walz L, Kays S K, Zimmer G, von Messling V. 2018. Neuraminidase-Inhibiting Antibody Titers Correlate with Protection from Heterologous Influenza Virus Strains of the Same Neuraminidase Subtype. J Virol 92.
19. Wohlbold T J, Nachbagauer R, Xu H, Tan G S, Hirsh A, Brokstad K A, Cox R J, Palese P, Krammer F. 2015. Vaccination with adjuvanted recombinant neuraminidase induces broad heterologous, but not heterosubtypic, cross-protection against influenza virus infection in mice. mBio 6:e02556.
20. Liu W, Zou P, Ding J, Lu Y, Chen Y H. 2005. Sequence comparison between the extracellular domain of M2 protein human and avian influenza A virus provides new information for bivalent influenza vaccine design. Microbes Infect 7:171-7.
21. Kim M C, Song J M, O E, Kwon Y M, Lee Y J, Compans R W, Kang S M. 2013. Virus-like particles containing multiple M2 extracellular domains confer improved cross-protection against various subtypes of influenza virus. Mol Ther 21:485-92.
22. Tang X, Yang Y, Xia X, Zhang C, Yang X, Song Y, Dai X, Wang M, Zhou D. 2017. Recombinant Adenoviruses Displaying Matrix 2 Ectodomain Epitopes on Their Fiber Proteins as Universal Influenza Vaccines. J Virol 91.
23. Berthoud T K, Hamill M, Lillie P J, Hwenda L, Collins K A, Ewer K J, Milicic A, Poyntz H C, Lambe T, Fletcher H A, Hill A V, Gilbert S C. 2011. Potent CD8+ T-cell immunogenicity in humans of a novel heterosubtypic influenza A vaccine, MVA-NP+M1. Clin Infect Dis 52:1-7.
24. Epstein S L, Kong W P, Misplon J A, Lo C Y, Tumpey T M, Xu L, Nabel G J. 2005. Protection against multiple influenza A subtypes by vaccination with highly conserved nucleoprotein. Vaccine 23:5404-10.
25. Nogales A, Baker S F, Domm W, Martinez-Sobrido L. 2016. Development and applications of single-cycle infectious influenza A virus (sciIAV). Virus Res 216:26-40.
26. Rossman J S, Lamb R A. 2011. Influenza virus assembly and budding. Virology 411:229-36.
27. Takizawa N, Momose F, Morikawa Y, Nomoto A. 2016. Influenza A Virus Hemagglutinin is Required for the Assembly of Viral Components Including Bundled vRNPs at the Lipid Raft. Viruses-Basel 8.
28. He X S, Holmes T H, Zhang C, Mahmood K, Kemble G W, Lewis D B, Dekker C L, Greenberg H B, Arvin A M. 2006. Cellular immune responses in children and adults receiving inactivated or live attenuated influenza vaccines. J Virol 80:11756-66.
29. Krammer F. 2019. The human antibody response to influenza A virus infection and vaccination. Nat Rev Immunol 19:383-397.
30. Wohlbold T J, Nachbagauer R, Xu H M, Tan G S, Hirsh A, Brokstad K A, Cox R J, Palese P, Krammer F. 2015. Vaccination with Adjuvanted Recombinant Neuraminidase Induces Broad Heterologous, but Not Heterosubtypic, Cross-Protection against Influenza Virus Infection in Mice. Mbio 6.
31. Harding A T, Heaton N S. 2018. Efforts to Improve the Seasonal Influenza Vaccine. Vaccines (Basel) 6.
32. Baker S F, Guo H, Albrecht R A, Garcia-Sastre A, Topham D J, Martinez-Sobrido L. 2013. Protection against lethal influenza with a viral mimic. J Virol 87:8591-605.
33. Nogales A, Baker S F, Domm W, Martinez-Sobrido L. 2016. Development and applications of single-cycle infectious influenza A virus (sciIAV). Virus Research 216:26-40.
34. Powell T J, Silk J D, Sharps J, Fodor E, Townsend A R M. 2012. Pseudotyped Influenza A Virus as a Vaccine for the Induction of Heterotypic Immunity. Journal of Virology 86:13397-13406.
35. Liu W C, Lin C Y, Tsou Y T, Jan J T, Wu S C. 2015. Cross-Reactive Neuraminidase-Inhibiting Antibodies Elicited by Immunization with Recombinant Neuraminidase Proteins of H5N1 and Pandemic H1N1 Influenza A Viruses. J Virol 89:7224-34.
36. Krammer F, Fouchier RAM, Eichelberger M C, Webby R J, Shaw-Saliba K, Wan H, Wilson P C, Compans R W, Skountzou I, Monto A S. 2018. NAction! How Can Neuraminidase-Based Immunity Contribute to Better Influenza Virus Vaccines? mBio 9.
37. Gotch F, McMichael A, Smith G, Moss B. 1987. Identification of viral molecules recognized by influenza-specific human cytotoxic T lymphocytes. J Exp Med 165:408-16.
38. Ansari A M, Ahmed A K, Matsangos A E, Lay F, Born L J, Marti G, Harmon J W, Sun Z. 2016. Cellular GFP Toxicity and Immunogenicity: Potential Confounders in in Vivo Cell Tracking Experiments. Stem Cell Rev Rep 12:553-559.
39. Chen Y Q, Lan L Y, Huang M, Henry C, Wilson P C. 2019. Hemagglutinin Stalk-Reactive Antibodies Interfere with Influenza Virus Neuraminidase Activity by Steric Hindrance. J Virol 93.
40. Harding A T, Heaton B E, Dumm R E, Heaton N S. 2017. Rationally Designed Influenza Virus Vaccines That Are Antigenically Stable during Growth in Eggs. mBio 8.
41. Heaton N S, Leyva-Grado V H, Tan G S, Eggink D, Hai R, Palese P. 2013. In vivo bioluminescent imaging of influenza a virus infection and characterization of novel cross-protective monoclonal antibodies. J Virol 87:8272-81.

### Example 2

In the following example, the inventors describe the generation of HA-negative IAV particles that express a heterologous viral antigen.

### Materials and Methods:

### Virus Rescue

Influenza viruses were rescued by transfecting HEK 293T cells with 8 plasmids that contain bicistronic expression cassettes to produce all 8 viral proteins and corresponding viral RNA. In the case of the NA only virus, segment 4 (which encodes the HA protein) was replaced with the mCherry ORF flanked by HA packaging signals. In the case of the RBD viruses, segment 4 was modified to express either RBD and/or sfGFP upstream of HA, separated by a 2A cleavage site. 0.5 μg of each plasmid was transfected into HEK 293T cells using TransIT LT-1 transfection reaction. Transfected cells were incubated at 37° C.+5% CO2 for 72 hours to produce virus. After 72 hours, cell supernatants were collected, filtered through a 0.45 μm filter, and applied to confluent monolayers of MDCK cells in the presence of 1 μg/mL TCPK-trypsin. Virus was allowed to propagate on these cells for 72 hours at 37° C. Cell supernatants were collected after 72 hours, and cellular debris was pelleted at 1,000×g for 10 minutes. The clarified supernatant was aliquoted and froze at −80° C.

### Generation of MDCK-RBD Cell Line

The receptor binding domain (RBD) of the spike protein of SARS-CoV-2 (SARS-CoV-2 RBD) was cloned into the pLEX lentiviral vector using standard restriction enzyme cloning techniques. HEK 293T cells were transfected with pLEX-RBD alongside the pMD.2G/pCMVR8.74 (which express the lentiviral Gag/Pol/VSV-G proteins) using polyethylenimine (PEI). Transfected cells were incubated at 37° C. for 72 hours to produce virus. After 72 hours, cell supernatant was collected, filtered through a 0.45 μm filter, and applied to confluent monolayers of MDCK cells. MDCKs were transduced with lentiviruses for 24 hours before beginning the selection process. To select for successfully transduced cells, tissue cultures were split into media containing 1 μg/mL puromycin.

### RBD Immunostaining

MDCK-RBD cells were plated in 6-well plates and grown to ˜90% confluence. Growth media was removed, and cells were fixed using 4% formaldehyde. RBD was detected using commercially available polyclonal antibodies (ProSci cat. no. 9087). Anti-rabbit-HRP secondary antibodies coupled with TrueBlue peroxidase substrate (SeraCare cat. no. 5510-0030) were used to stain cells detected by the RBD antibodies.

### ELISAs

For whole-virus ELISAs, 1×10⁶ PFUs of virus were coated onto wells using carbonate coating buffer (50 mM carbonate, pH 9.4). During the coating step, plates were incubated at 4° C. overnight. Plates were washed with PBS and probed using anti-RBD antibodies for 1 hour at room temperature. ELISAs were developed using anti-rabbit-HRP secondary antibodies coupled with 1-Step TMB-Ultra ELISA substrate (Thermo cat. no. 34029). Colorimetric reactions were quenched using 1M H₂SO₄ and absorbances were measured at 450 nm.

### Hemagglutinin Assays

Virus was added to 96-well v-bottom plates and diluted in 2-fold steps using PBS. Turkey blood was diluted to 2.5% in PBS and added to each well containing virus. Plates were incubated at 4° C. for at least 1 hour.

### Plaque Assays

Virus was diluted in PBS in 10-fold steps to 10⁻⁹. Diluted virus was added to confluent monolayers of MDCKs and incubated at 37° C. for 1 hour. After 1 hour, virus was aspirated from cells and agar overlays containing 1 μg/mL TCPK-trypsin were applied to each well. Plates were incubated at 37° C. until plaques reached a suitable size.

### Results:

### Cell Lines Expressing Heterologous Viral Antigens can be Readily Generated

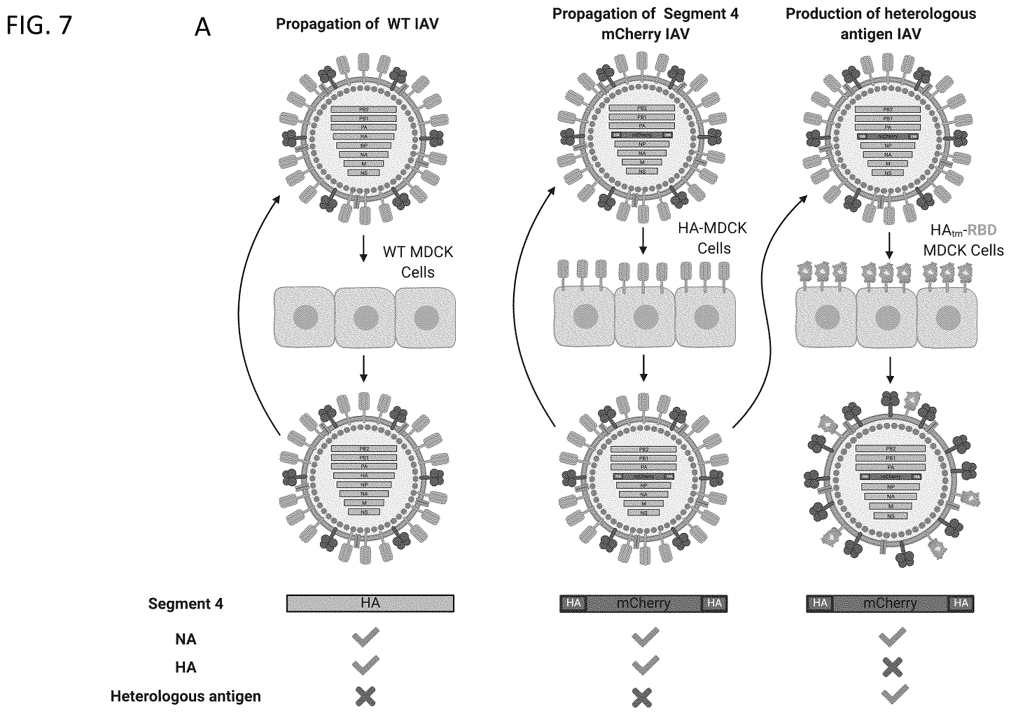

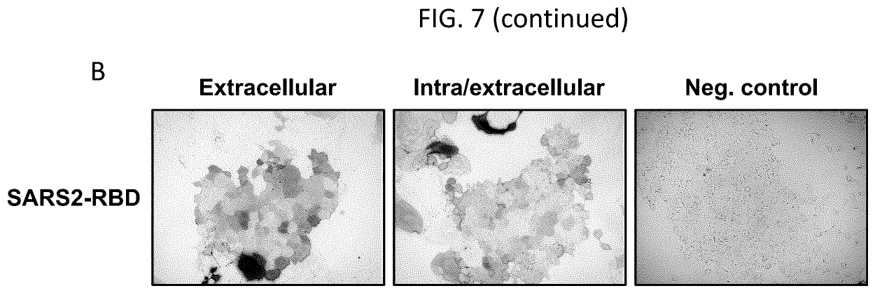

In the IAV generation strategy depicted in FIG. 7, the HA protein on the viral particle surface is replaced with an antigen that is a desired vaccine target, e.g., the receptor binding domain (RBD) of the spike protein of SARS-CoV-2 (SARS-CoV-2 RBD). As proof-of-concept, we engineered an MDCK cell line to stably express the SARS-CoV-2 RBD, termed HA_{TM}-RBD MDCK cells. To generate IAV particles that comprise HA_{TM}-RBD, mCherry IAV particles can be generated in a first cell line that expresses HA and then used to infect the HA_{TM}-RBD MDCK cells (FIG. 7A).

We confirmed expression of the RBD in the HA_{TM}-RBD MDCK cells via immunostaining using anti-SARS-CoV-2 RBD antibodies on fixed cells (FIG. 7B). While expression levels appear to be heterogenous across the culture, the foreign epitope is expressed to some degree in almost all cells. High levels of homogenous expression could easily be achieved via clonal selection from this polyclonal cell line. Importantly, constitutive expression of the RBD is well tolerated by these cells. These data demonstrate the feasibility of this strategy as a vaccine development approach.

### Heterologous Antigens can be Stably Inserted in the IAV Genome

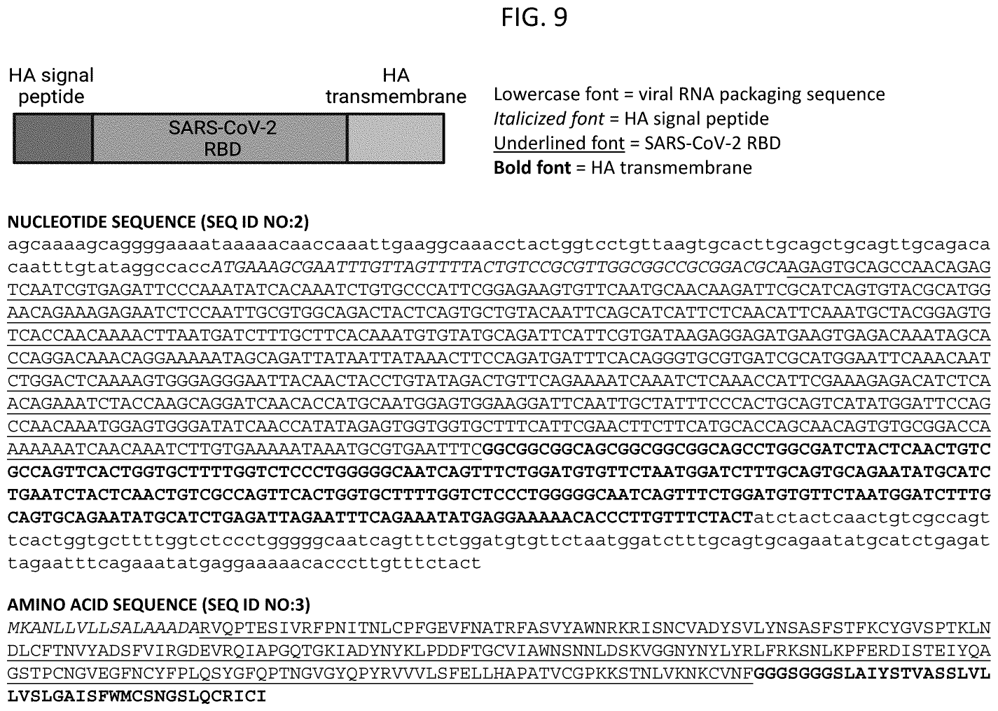

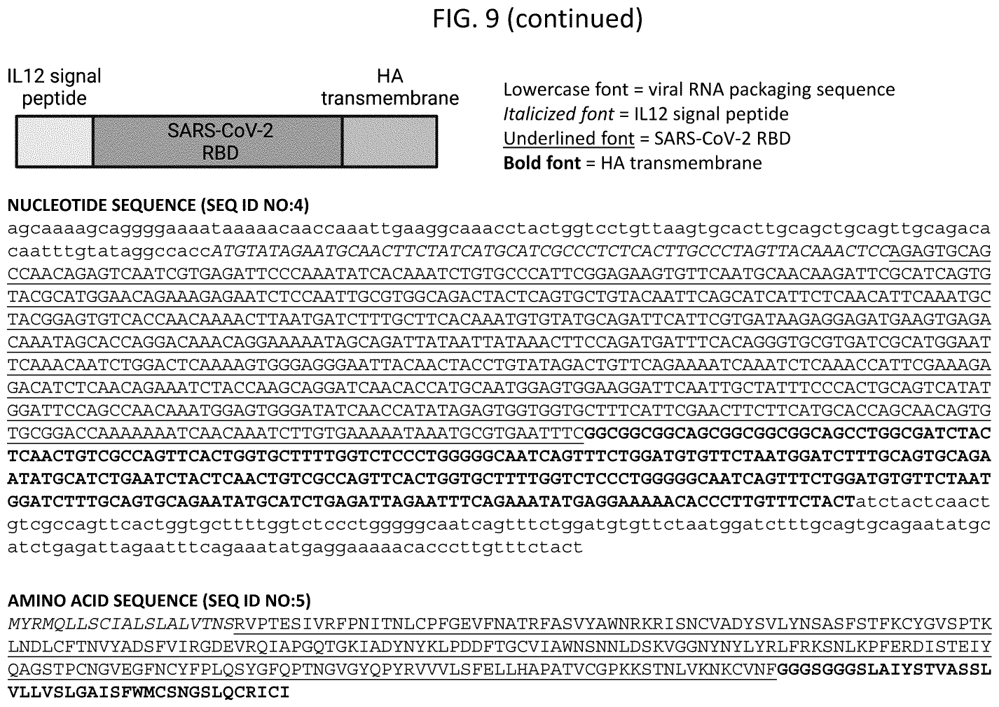

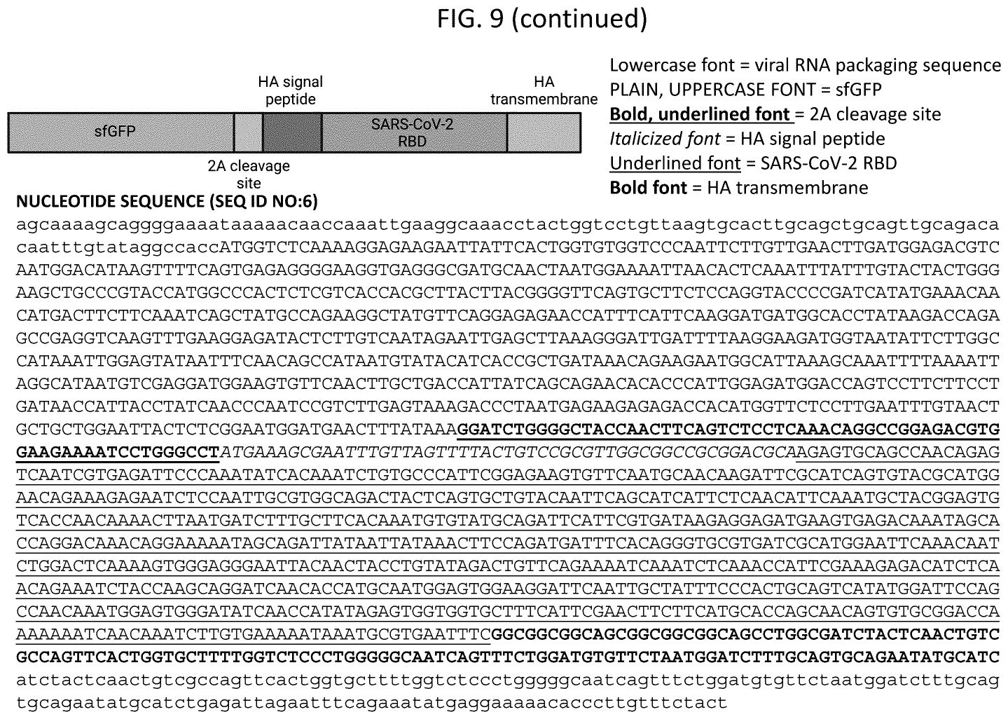

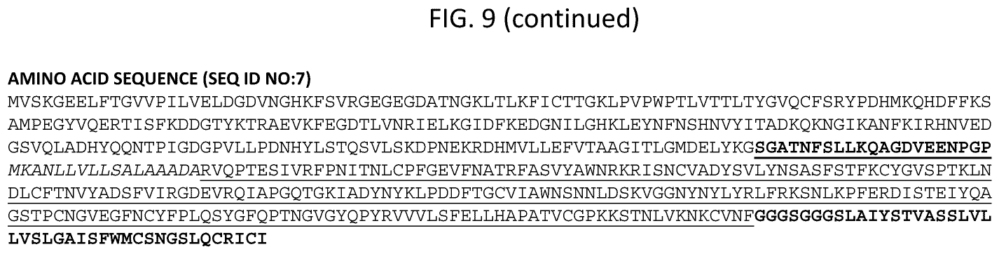

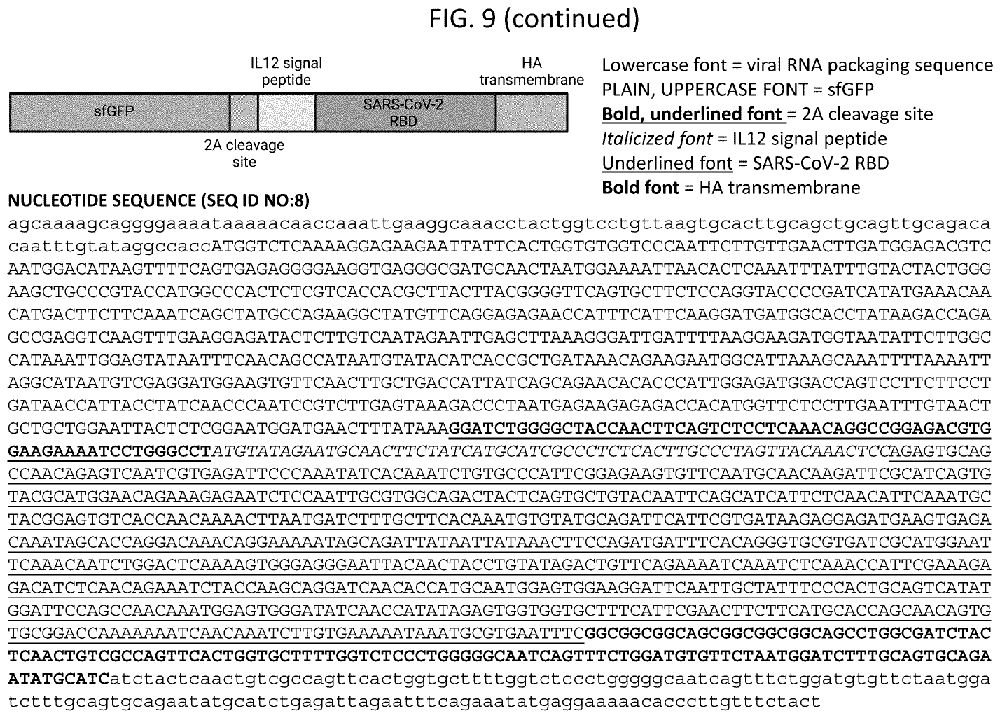

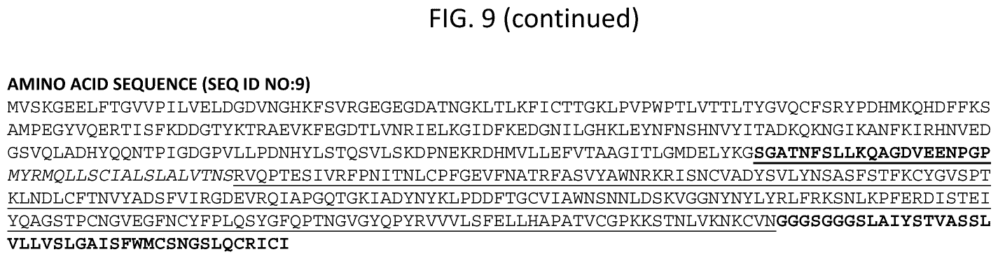

The IAV generation strategy depicted in FIG. 8 focuses on genetic engineering of the viral genome as opposed to the genetic engineering of cells. In this approach, segment 4 of the IAV genome, which encodes HA, is modified to include additional coding capacity that allows for expression of a foreign epitope (e.g. SARS-CoV-2 RBD)(FIG. 8A). The modified HA constructs that were tested are depicted schematically in FIG. 8B, and the nucleotide and amino acid sequences of these constructs are depicted in FIG. 9 and provided as SEQ ID NOs:2-9. We were able to successfully rescue viruses containing these modified genome segments (FIG. 8C). These modified segments appear to be well-tolerated by the virus, as total particle and infectious particle concentrations were sufficiently high (FIG. 8D, E). Importantly, these viruses package the foreign epitope and are detectable via ELISA (FIG. 8F). These data conclusively show that IAVs can be modified to express a foreign epitope with only a modest reduction in viral fitness.

### Example 3

In the following example, the inventors describe the generation of IAV particles that express a headless HA protein.

### Results:

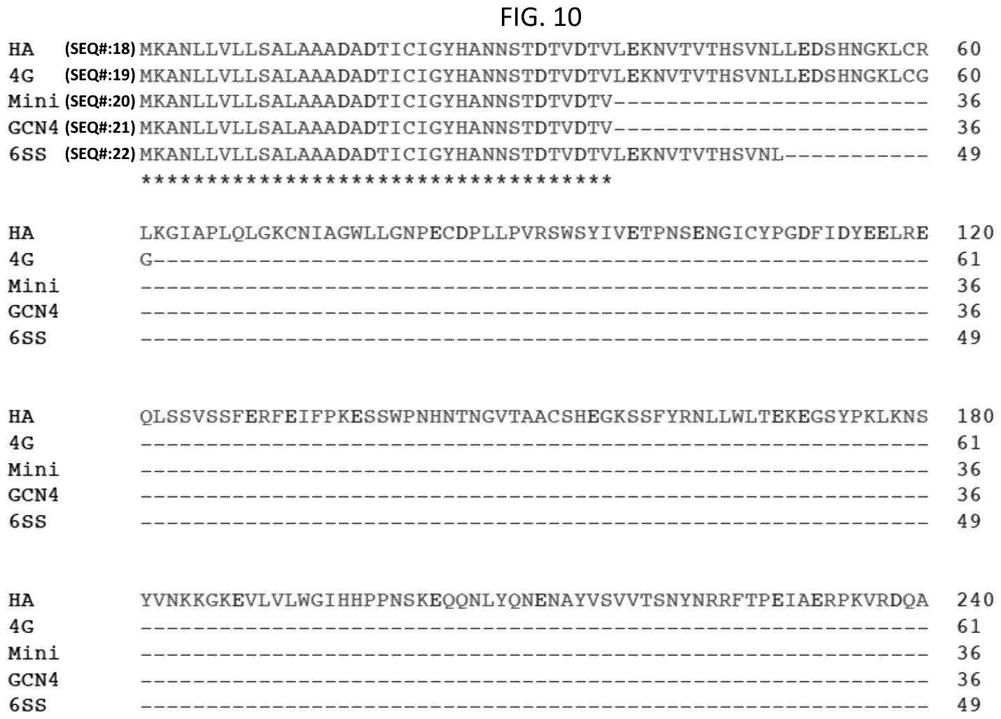

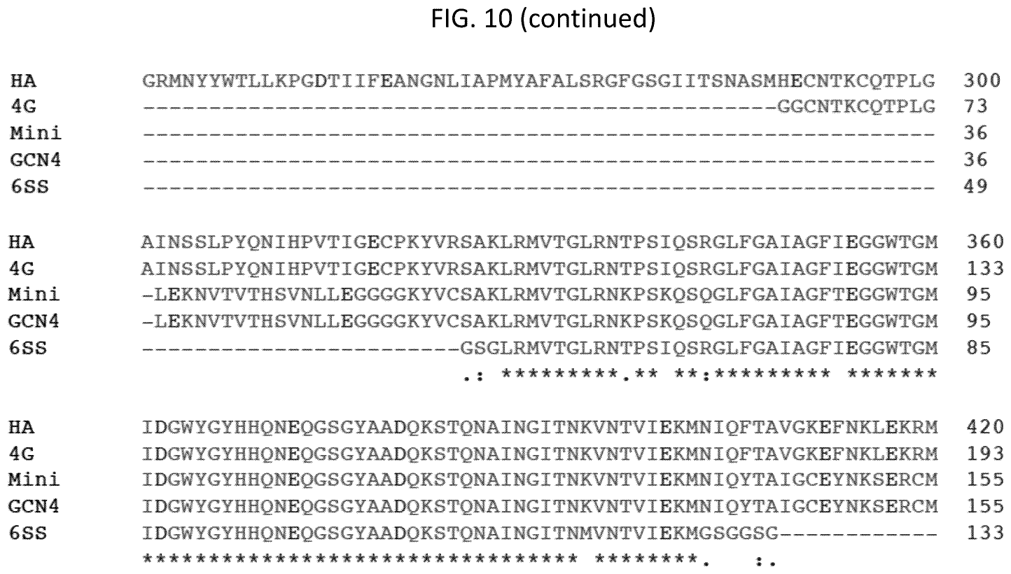

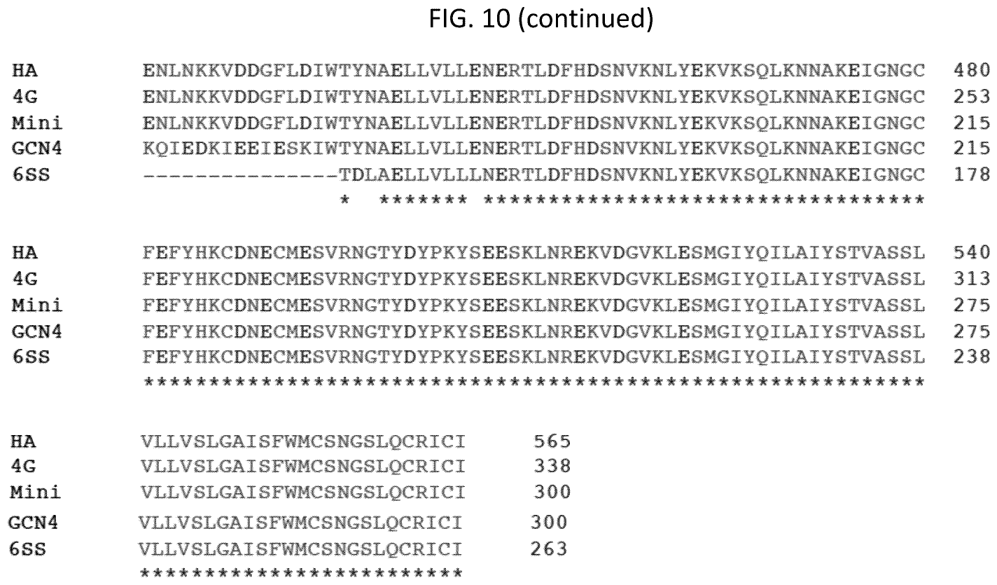

FIG. 10 shows amino acid sequences of the headless HA (4G, Mini, GCN4, 6SS) designs aligned to wild-type HA.

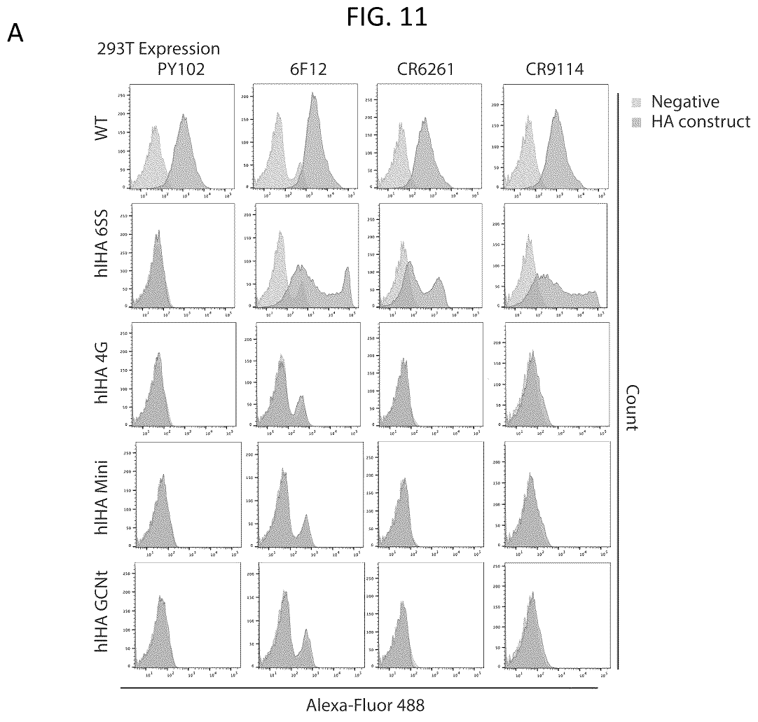

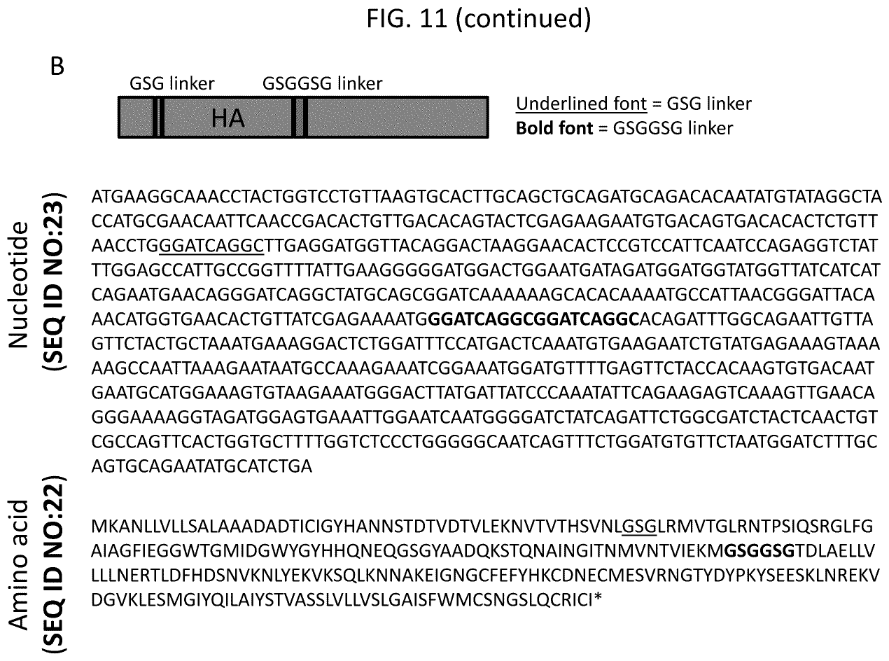

FIG. 11 shows headless HA design validation. (A) 293T cells were transfected with the designed headless HA expression plasmids, and then stained with primary antibodies that specifically recognize the head (PY102) or stalk (6F12, CR6261, CR9914) of hemagglutinin. Samples were then stained with secondary antibody conjugated to AlexaFluor-488. Flow cytometry was used to measure primary antibody binding. Wild-type HA was used as a positive control. Of the 4 headless HA designs, only cells expressing the 6SS design were positive for stalk antibody binding and negative for head antibody binding. (B) Schematic of the 6SS headless HA design, along with the nucleotide and amino acid sequences.

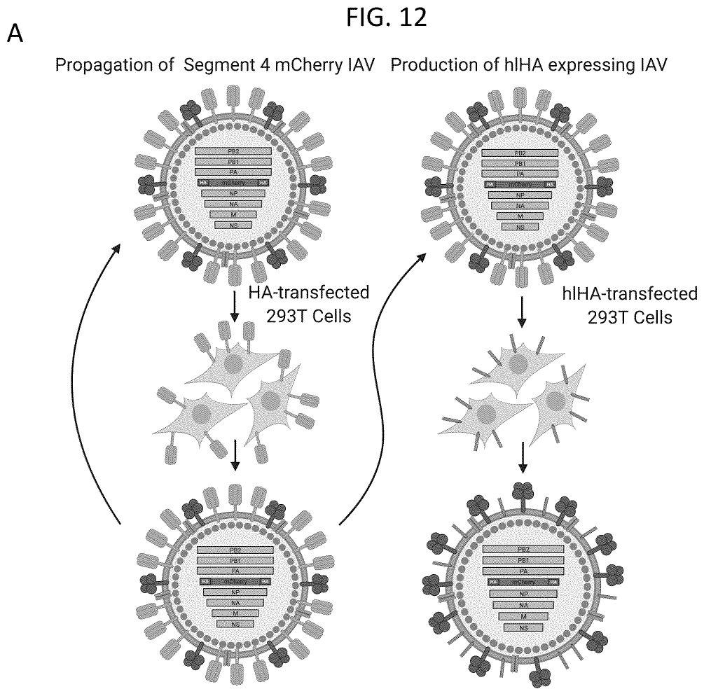

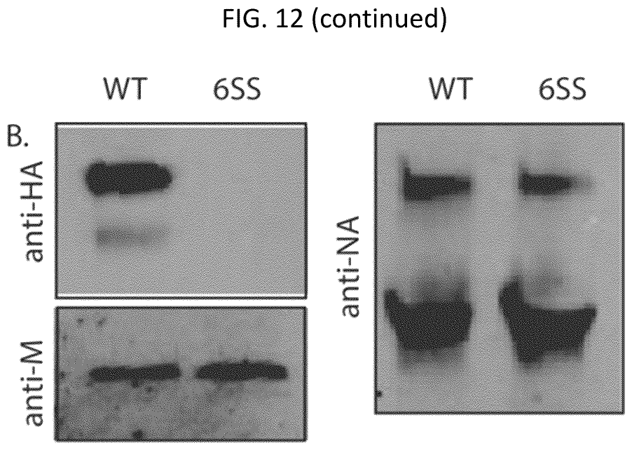

FIG. 12 shows the generation of IAV particles that comprise a headless HA (hlHA) protein. (A) Schematic of headless HA virus propagation strategy. 293T cells are transfected with wild-type HA expression plasmid and then infected with an influenza virus encoding mCherry in place of HA in segment 4 (PR8-deltaHA-mCherry). Supernatant containing the newly propagated virus (PR8-deltaHA-mCherry) is then placed on 293T cells transfected with the 6SS headless HA expression construct. The supernatant contains virus expressing the headless HA (PR8-deltaHA-mCherry-6SShlHA). (B) Wild-type and 6SS headless HA viruses were concentrated and western blotting was used to detect viral protein expression. HA protein can be detected in concentrated wild-type virus but not the 6SS headless virus. Both the M and NA proteins can be detected for both viruses.

FIG. 13 demonstrates that mice vaccinated with 6SS generate higher antibody responses against hlHA, NA only viruses. Mice were vaccinated with inactivated wild-type PR8, 6SS headless HA PR8, a PR8 virus with surface expression of GFP in place of HA, or a BSA control. ELISA was used to measure serum reactivity against 6SS headless HA virus (left) and a virus that has all viral proteins except HA (right) as a proof of concept.

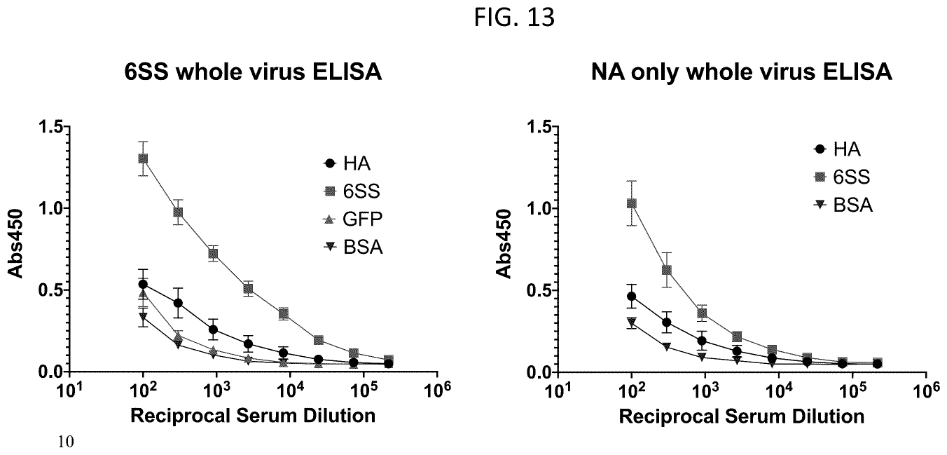

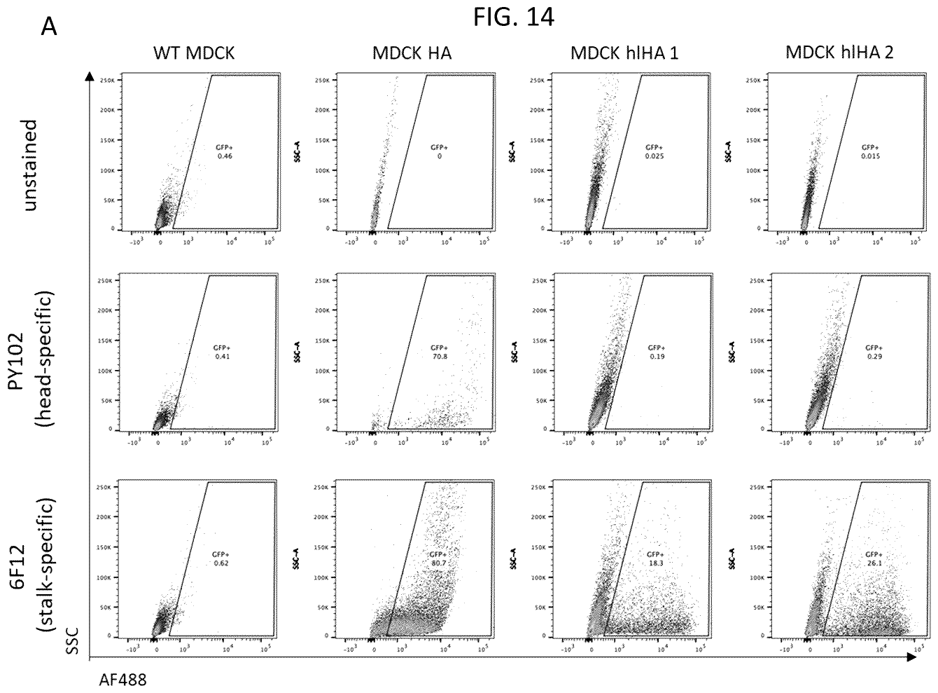

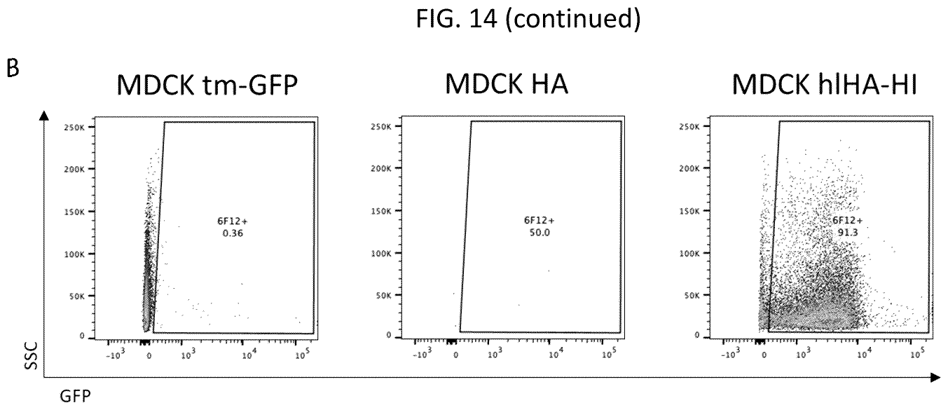

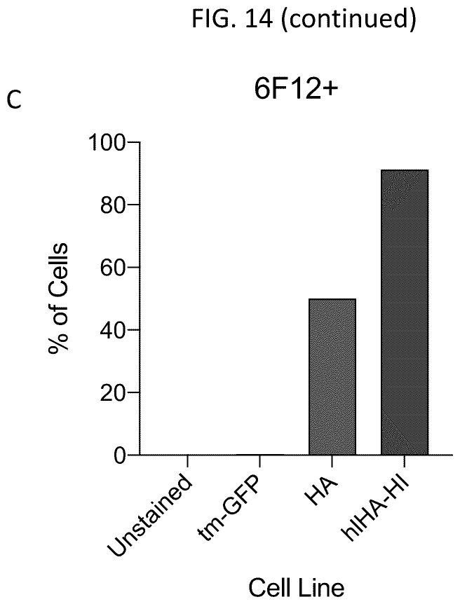

FIG. 14 demonstrates generation of stable cell line expressing hlHA for virus propagation. (A) MDCK cells were transduced with lentivirus packaging the 6SS headless HA construct to generate cell lines with stable expression of headless HA for virus propagation. For validation, 6SS headless HA MDCK cells along with wild-type MDCK cells and MDCK cells stably expressing wild-type PR8 HA were stained with primary antibodies targeting the head or stalk of hemagglutinin. Samples were then stained with secondary antibody conjugated to AF488 and flow cytometry was used to measure primary antibody binding. A population of cells in both headless HA MDCK cell lines positive for stalk staining. (B,C) FACS was used to collect MDCK cells with high expression of 6SS headless HA (based on antibody staining). The collected cells were expanded, and flow cytometry was used to determine the percentage of cells with expression of 6SS headless HA. MDCK cells expressing wild-type PR8 HA or GFP were used as a control.

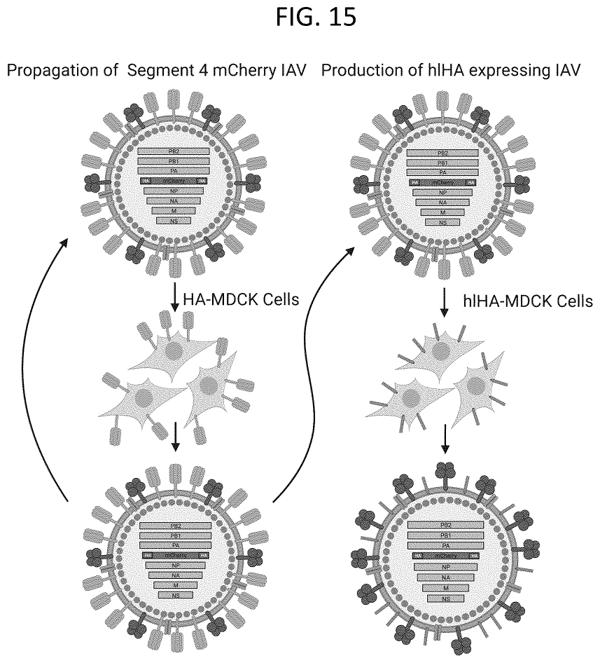

FIG. 15 shows the generation of IAV particles that comprise hlHA via propagation on hlHA-MDCK cells. MDCK cells with stable expression of PR8 HA are infected with an influenza virus encoding mCherry in place of HA in segment 4 (PR8-deltaHA-mCherry). Supernatant containing the newly propagated virus (PR8-deltaHA-mCherry) is then placed on MDCK cells with stable expression of 6SS headless HA. The supernatant contains virus expressing the headless HA (PR8-deltaHA-mCherry-6SShlHA).

What is claimed:

1. An influenza viral particle that comprises a modified hemagglutinin (HA) protein comprising the transmembrane domain of HA and the cytoplasmic tail of HA, wherein the modification in the HA gene renders the virus replication incompetent.

2. The viral particle of claim 1, wherein the modification in the HA gene is removal of the head domain of HA.

3. The viral particle of claim 1, wherein the modified HA protein comprises at least the 5′ 99 nucleotides and the 3′ 150 nucleotides encoding portions of the HA protein of the influenza virus.

4. The viral particle of claim 3, wherein the ATG codons in the 3′ terminal nucleotide region are mutated to TTG.

5. The viral particle of claim 1, wherein the modified HA protein comprises SEQ ID NO: 10 or a sequence with 90% identity to SEQ ID NO: 10.

6. The viral particle of any one of claims 1-5, wherein the modified HA protein further comprises a heterologous protein, and wherein the heterologous protein is present on the surface of the viral particle.

7. The viral particle of claim 6, wherein the heterologous protein is a viral antigen.

8. The viral particle of claim 7, wherein the viral antigen is from SARS-CoV-2.

9. The viral particle of claim 8, wherein the viral antigen is the receptor binding domain (RBD) of the spike protein.

10. The viral particle of claim 9, wherein the viral antigen is SEQ ID NO:11 or has 90% identity to SEQ ID NO: 11.

11. The viral particle of any one of claims 1-5, wherein the modified HA protein further comprises the stalk domain of HA, and wherein the stalk domain is present on the surface of the viral particle.

12. The viral particle of claim 11, wherein the stalk domain is selected from the group consisting of SEQ ID NO:24, a sequence with 90% identity to SEQ ID NO: 24, SEQ ID NO:25 and a sequence having 90% identity to SEQ ID NO: 25.

13. A vaccine formulation comprising the viral particle of any one of the preceding claims and a pharmaceutically acceptable carrier.

14. A method for producing the viral particle of any one of claims 1-12, the method comprising:
a) modifying the HA gene within segment 4 of the genome of an influenza virus in a manner that renders the virus replication incompetent;
b) transfecting the modified genome into a first cell line that expresses a wild-type HA protein on its surface;
c) culturing the transfected first cell line to produce viral particles that comprise the wild-type HA protein and the modified segment 4 of step (a);
d) infecting a second cell line that expresses a modified HA protein comprising the transmembrane domain of HA and the cytoplasmic tail of HA with the viral particles produced in step (c);
e) culturing the infected second cell line to produce replication incompetent viral particles that comprise the modified HA protein.

15. A method for producing the viral particle of any one of claims 1-12, the method comprising:
a) modifying the HA gene within segment 4 of the genome of an influenza virus to encode a modified HA protein comprising the transmembrane domain of HA and the cytoplasmic tail of HA, thereby rendering the virus replication incompetent;
b) transfecting the modified genome into a first cell line that expresses a wild-type HA protein on its surface;
c) culturing the transfected first cell line to produce viral particles that comprise the wild-type HA protein and the modified segment 4 of step (a);
d) infecting a second cell line that does not express HA with the viral particles produced in step (c);
e) culturing the infected second cell line to produce replication incompetent viral particles that comprise the modified HA protein.

16. A method for inducing an immune response in a subject, the method comprising: administering the viral particle of any one of claims 1-12 or the vaccine formulation of claim 13 to the subject.

17. The method of claim 16, wherein the immune response provides protection against a heterologous virus.

18. The method of claim 16 or 17, wherein the viral particle is administered at least twice.

19. The method of any one of claims 16-18, wherein the viral particle is administered intramuscularly.

20. The method of any one of claims 16-19, wherein the subject is a human.

21. An influenza-susceptible cell line that expresses a modified HA protein comprising the transmembrane domain of HA and the cytoplasmic tail of HA, but is modified to not express the head domain of HA.

22. The cell line of claim 21, wherein the modified HA protein further comprises a heterologous protein, and wherein the heterologous protein is present on the surface of the cells.

23. The cell line of claim 22, wherein the heterologous protein is a viral antigen.

24. The cell line of claim 23, wherein the viral antigen is from SARS-CoV-2.

25. The cell line of claim 24, wherein the viral antigen is the receptor binding domain (RBD) of the spike protein.

26. The cell line of claim 25, wherein the viral antigen is SEQ ID NO:11 or has 90% identity to SEQ ID NO: 11.

27. The cell line of claim 21, wherein the modified HA protein further comprises the stalk domain of HA, and wherein the stalk domain is present on the surface of the viral particle.

28. The cell line of claim 27, wherein the stalk domain is selected from the group consisting of SEQ ID NO:24, a sequence with 90% identity to SEQ ID NO: 24, SEQ ID NO:25 and a sequence having 90% identity to SEQ ID NO: 25.

29. The cell line of any one of claims 21-28, wherein the modified HA protein is expressed from a plasmid.

30. The cell line of any one of claims 21-28, wherein the modified HA protein is expressed from a stably integrated gene.


SEQUENCE LISTING (28 sequences)

SEQ ID NO: 1 - DNA, 918 bp, Artificial Sequence
  Synthetic - Fusion protein comprising GFP, HA transmembrane domain, and HA cytoplasmic tail
atgtatagaatgcaacttctatcatgcatcgccctctcacttgccctagttacaaactccatggtctcaaaaggagaagaattattcactggtgtggtcccaattcttgttgaacttgatggagacgtcaatggacataagttttcagtgagaggggaaggtgagggcgatgcaactaatggaaaattaacactcaaatttatttgtactactgggaagctgcccgtaccatggcccactctcgtcaccacgcttacttacggggttcagtgcttctccaggtaccccgatcatatgaaacaacatgacttcttcaaatcagctatgccagaaggctatgttcaggagagaaccatttcattcaaggatgatggcacctataagaccagagccgaggtcaagtttgaaggagatactcttgtcaatagaattgagcttaaagggattgattttaaggaagatggtaatattcttggccataaattggagtataatttcaacagccataatgtatacatcaccgctgataaacagaagaatggcattaaagcaaattttaaaattaggcataatgtcgaggatggaagtgttcaacttgctgaccattatcagcagaacacacccattggagatggaccagtccttcttcctgataaccattacctatcaacccaatccgtcttgagtaaagaccctaatgagaagagagaccacatggttctccttgaatttgtaactgctgctggaattactctcggaatggatgaactttataaaggcggcggcagcggcggcggcagccagattctggcgatctactcaactgtcgccagttcactggtgcttttggtctccctgggggcaatcagtttctggatgtgttctaatggatctttgcagtgcagaatatgcatctga

SEQ ID NO: 2 - DNA, 1110 bp, Artificial Sequence
  Synthetic - Fusion protein comprising HA signal peptide, SARS-CoV-2 RBD, and HA transmembrane domain/cytoplasmic tail
agcaaaagcaggggaaaataaaaacaaccaaattgaaggcaaacctactggtcctgttaagtgcacttgcagctgcagttgcagacacaatttgtataggccaccatgaaagcgaatttgttagttttactgtccgcgttggcggccgcggacgcaagagtgcagccaacagagtcaatcgtgagattcccaaatatcacaaatctgtgcccattcggagaagtgttcaatgcaacaagattcgcatcagtgtacgcatggaacagaaagagaatctccaattgcgtggcagactactcagtgctgtacaattcagcatcattctcaacattcaaatgctacggagtgtcaccaacaaaacttaatgatctttgcttcacaaatgtgtatgcagattcattcgtgataagaggagatgaagtgagacaaatagcaccaggacaaacaggaaaaatagcagattataattataaacttccagatgatttcacagggtgcgtgatcgcatggaattcaaacaatctggactcaaaagtgggagggaattacaactacctgtatagactgttcagaaaatcaaatctcaaaccattcgaaagagacatctcaacagaaatctaccaagcaggatcaacaccatgcaatggagtggaaggattcaattgctatttcccactgcagtcatatggattccagccaacaaatggagtgggatatcaaccatatagagtggtggtgctttcattcgaacttcttcatgcaccagcaacagtgtgcggaccaaaaaaatcaacaaatcttgtgaaaaataaatgcgtgaatttcggcggcggcagcggcggcggcagcctggcgatctactcaactgtcgccagttcactggtgcttttggtctccctgggggcaatcagtttctggatgtgttctaatggatctttgcagtgcagaatatgcatctgaatctactcaactgtcgccagttcactggtgcttttggtctccctgggggcaatcagtttctggatgtgttctaatggatctttgcagtgcagaatatgcatctgagattagaatttcagaaatatgaggaaaaacacccttgtttctact

SEQ ID NO: 3 - protein, 284 aa, Artificial Sequence
  Synthetic - Fusion protein comprising HA signal peptide, SARS-CoV-2 RBD, and HA transmembrane domain/cytoplasmic tail
MKANLLVLLSALAAADARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGGSGGGSLAIYSTVASSLVLLVSLGAISFWMCSNGSLQCRICI

SEQ ID NO: 4 - DNA, 1119 bp, Artificial Sequence
  Synthetic - Fusion protein comprising IL12 signal peptide, SARS-CoV-2 RBD, and HA transmembrane domain/ cytoplasmic tail
agcaaaagcaggggaaaataaaaacaaccaaattgaaggcaaacctactggtcctgttaagtgcacttgcagctgcagttgcagacacaatttgtataggccaccatgtatagaatgcaacttctatcatgcatcgccctctcacttgccctagttacaaactccagagtgcagccaacagagtcaatcgtgagattcccaaatatcacaaatctgtgcccattcggagaagtgttcaatgcaacaagattcgcatcagtgtacgcatggaacagaaagagaatctccaattgcgtggcagactactcagtgctgtacaattcagcatcattctcaacattcaaatgctacggagtgtcaccaacaaaacttaatgatctttgcttcacaaatgtgtatgcagattcattcgtgataagaggagatgaagtgagacaaatagcaccaggacaaacaggaaaaatagcagattataattataaacttccagatgatttcacagggtgcgtgatcgcatggaattcaaacaatctggactcaaaagtgggagggaattacaactacctgtatagactgttcagaaaatcaaatctcaaaccattcgaaagagacatctcaacagaaatctaccaagcaggatcaacaccatgcaatggagtggaaggattcaattgctatttcccactgcagtcatatggattccagccaacaaatggagtgggatatcaaccatatagagtggtggtgctttcattcgaacttcttcatgcaccagcaacagtgtgcggaccaaaaaaatcaacaaatcttgtgaaaaataaatgcgtgaatttcggcggcggcagcggcggcggcagcctggcgatctactcaactgtcgccagttcactggtgcttttggtctccctgggggcaatcagtttctggatgtgttctaatggatctttgcagtgcagaatatgcatctgaatctactcaactgtcgccagttcactggtgcttttggtctccctgggggcaatcagtttctggatgtgttctaatggatctttgcagtgcagaatatgcatctgagattagaatttcagaaatatgaggaaaaacacccttgtttctact

SEQ ID NO: 5 - protein, 286 aa, Artificial Sequence
  Synthetic - Fusion protein comprising IL12 signal peptide, SARS-CoV-2 RBD, and HA transmembrane domain/ cytoplasmic tail
MYRMQLLSCIALSLALVTNSRVPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGGSGGGSLAIYSTVASSLVLLVSLGAISFWMCSNGSLQCRICI

SEQ ID NO: 6 - DNA, 1890 bp, Artificial Sequence
  Synthetic - Fusion protein comprising sfGFP, 2A cleavage site, HA signal peptide, SARS-CoV-2 RBD, and HA transmembrane domain/cytoplasmic tail
agcaaaagcaggggaaaataaaaacaaccaaattgaaggcaaacctactggtcctgttaagtgcacttgcagctgcagttgcagacacaatttgtataggccaccatggtctcaaaaggagaagaattattcactggtgtggtcccaattcttgttgaacttgatggagacgtcaatggacataagttttcagtgagaggggaaggtgagggcgatgcaactaatggaaaattaacactcaaatttatttgtactactgggaagctgcccgtaccatggcccactctcgtcaccacgcttacttacggggttcagtgcttctccaggtaccccgatcatatgaaacaacatgacttcttcaaatcagctatgccagaaggctatgttcaggagagaaccatttcattcaaggatgatggcacctataagaccagagccgaggtcaagtttgaaggagatactcttgtcaatagaattgagcttaaagggattgattttaaggaagatggtaatattcttggccataaattggagtataatttcaacagccataatgtatacatcaccgctgataaacagaagaatggcattaaagcaaattttaaaattaggcataatgtcgaggatggaagtgttcaacttgctgaccattatcagcagaacacacccattggagatggaccagtccttcttcctgataaccattacctatcaacccaatccgtcttgagtaaagaccctaatgagaagagagaccacatggttctccttgaatttgtaactgctgctggaattactctcggaatggatgaactttataaaggatctggggctaccaacttcagtctcctcaaacaggccggagacgtggaagaaaatcctgggcctatgaaagcgaatttgttagttttactgtccgcgttggcggccgcggacgcaagagtgcagccaacagagtcaatcgtgagattcccaaatatcacaaatctgtgcccattcggagaagtgttcaatgcaacaagattcgcatcagtgtacgcatggaacagaaagagaatctccaattgcgtggcagactactcagtgctgtacaattcagcatcattctcaacattcaaatgctacggagtgtcaccaacaaaacttaatgatctttgcttcacaaatgtgtatgcagattcattcgtgataagaggagatgaagtgagacaaatagcaccaggacaaacaggaaaaatagcagattataattataaacttccagatgatttcacagggtgcgtgatcgcatggaattcaaacaatctggactcaaaagtgggagggaattacaactacctgtatagactgttcagaaaatcaaatctcaaaccattcgaaagagacatctcaacagaaatctaccaagcaggatcaacaccatgcaatggagtggaaggattcaattgctatttcccactgcagtcatatggattccagccaacaaatggagtgggatatcaaccatatagagtggtggtgctttcattcgaacttcttcatgcaccagcaacagtgtgcggaccaaaaaaatcaacaaatcttgtgaaaaataaatgcgtgaatttcggcggcggcagcggcggcggcagcctggcgatctactcaactgtcgccagttcactggtgcttttggtctccctgggggcaatcagtttctggatgtgttctaatggatctttgcagtgcagaatatgcatcatctactcaactgtcgccagttcactggtgcttttggtctccctgggggcaatcagtttctggatgtgttctaatggatctttgcagtgcagaatatgcatctgagattagaatttcagaaatatgaggaaaaacacccttgtttctact

SEQ ID NO: 7 - protein, 545 aa, Artificial Sequence
  Synthetic - Fusion protein comprising sfGFP, 2A cleavage site, HA signal peptide, SARS-CoV-2 RBD, and HA transmembrane domain/cytoplasmic tail
MVSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITLGMDELYKGSGATNFSLLKQAGDVEENPGPMKANLLVLLSALAAADARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGGSGGGSLAIYSTVASSLVLLVSLGAISFWMCSNGSLQCRICI

SEQ ID NO: 8 - DNA, 1899 bp, Artificial Sequence
  Synthetic - Fusion protein comprising sfGFP, 2A cleavage site, IL12 signal peptide, SARS-CoV-2 RBD, and HA transmembrane domain/cytoplasmic tail
agcaaaagcaggggaaaataaaaacaaccaaattgaaggcaaacctactggtcctgttaagtgcacttgcagctgcagttgcagacacaatttgtataggccaccatggtctcaaaaggagaagaattattcactggtgtggtcccaattcttgttgaacttgatggagacgtcaatggacataagttttcagtgagaggggaaggtgagggcgatgcaactaatggaaaattaacactcaaatttatttgtactactgggaagctgcccgtaccatggcccactctcgtcaccacgcttacttacggggttcagtgcttctccaggtaccccgatcatatgaaacaacatgacttcttcaaatcagctatgccagaaggctatgttcaggagagaaccatttcattcaaggatgatggcacctataagaccagagccgaggtcaagtttgaaggagatactcttgtcaatagaattgagcttaaagggattgattttaaggaagatggtaatattcttggccataaattggagtataatttcaacagccataatgtatacatcaccgctgataaacagaagaatggcattaaagcaaattttaaaattaggcataatgtcgaggatggaagtgttcaacttgctgaccattatcagcagaacacacccattggagatggaccagtccttcttcctgataaccattacctatcaacccaatccgtcttgagtaaagaccctaatgagaagagagaccacatggttctccttgaatttgtaactgctgctggaattactctcggaatggatgaactttataaaggatctggggctaccaacttcagtctcctcaaacaggccggagacgtggaagaaaatcctgggcctatgtatagaatgcaacttctatcatgcatcgccctctcacttgccctagttacaaactccagagtgcagccaacagagtcaatcgtgagattcccaaatatcacaaatctgtgcccattcggagaagtgttcaatgcaacaagattcgcatcagtgtacgcatggaacagaaagagaatctccaattgcgtggcagactactcagtgctgtacaattcagcatcattctcaacattcaaatgctacggagtgtcaccaacaaaacttaatgatctttgcttcacaaatgtgtatgcagattcattcgtgataagaggagatgaagtgagacaaatagcaccaggacaaacaggaaaaatagcagattataattataaacttccagatgatttcacagggtgcgtgatcgcatggaattcaaacaatctggactcaaaagtgggagggaattacaactacctgtatagactgttcagaaaatcaaatctcaaaccattcgaaagagacatctcaacagaaatctaccaagcaggatcaacaccatgcaatggagtggaaggattcaattgctatttcccactgcagtcatatggattccagccaacaaatggagtgggatatcaaccatatagagtggtggtgctttcattcgaacttcttcatgcaccagcaacagtgtgcggaccaaaaaaatcaacaaatcttgtgaaaaataaatgcgtgaatttcggcggcggcagcggcggcggcagcctggcgatctactcaactgtcgccagttcactggtgcttttggtctccctgggggcaatcagtttctggatgtgttctaatggatctttgcagtgcagaatatgcatcatctactcaactgtcgccagttcactggtgcttttggtctccctgggggcaatcagtttctggatgtgttctaatggatctttgcagtgcagaatatgcatctgagattagaatttcagaaatatgaggaaaaacacccttgtttctact

SEQ ID NO: 9 - protein, 547 aa, Artificial Sequence
  Synthetic - Fusion protein comprising sfGFP, 2A cleavage site, IL12 signal peptide, SARS-CoV-2 RBD, and HA transmembrane domain/cytoplasmic tail
MVSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITLGMDELYKGSGATNFSLLKQAGDVEENPGPMYRMQLLSCIALSLALVTNSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNGGGSGGGSLAIYSTVASSLVLLVSLGAISFWMCSNGSLQCRICI

SEQ ID NO: 10 - protein, 36 aa, Artificial Sequence
  Synthetic - HA transmembrane domain/cytoplasmic tail
LAIYSTVASSLVLLVSLGAISFWMCSNGSLQCRICI

SEQ ID NO: 11 - protein, 223 aa, Artificial Sequence
  Synthetic - SARS-CoV-2 RBD
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 12 - DNA, 105 bp, Artificial Sequence
  Synthetic - 5′ influenza virus packaging signal
agcaaaagcaggggaaaataaaaacaaccaaattgaaggcaaacctactggtcctgttaagtgcacttgcagctgcagttgcagacacaatttgtataggccacc

SEQ ID NO: 13 - DNA, 150 bp, Artificial Sequence
  Synthetic - 3′ influenza virus packaging signal
atctactcaactgtcgccagttcactggtgcttttggtctccctgggggcaatcagtttctggatgtgttctaatggatctttgcagtgcagaatatgcatctgagattagaatttcagaaatatgaggaaaaacacccttgtttctact

SEQ ID NO: 14 - protein, 17 aa, Artificial Sequence
  Synthetic - HA signal peptide
MKANLLVLLSALAAADA

SEQ ID NO: 15 - protein, 20 aa, Artificial Sequence
  Synthetic - IL12 signal peptide
MYRMQLLSCIALSLALVTNS

SEQ ID NO: 16 - protein, 21 aa, Artificial Sequence
  Synthetic - 2A cleavage site
SGATNFSLLKQAGDVEENPGP

SEQ ID NO: 17 - protein, 240 aa, Artificial Sequence
  Synthetic - Superfolder GFP (sfGFP)
MVSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITLGMDELYKG

SEQ ID NO: 18 - protein, 565 aa, Influenza A virus
  MISC_FEATURE (1)..(58): Stalk domain
  MISC_FEATURE (59)..(291): Head domain
  MISC_FEATURE (292)..(565): Stalk domain
MKANLLVLLSALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNTPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 19 - protein, 338 aa, Artificial Sequence
  Synthetic - 4G headless HA
MKANLLVLLSALAAADADTICIGYHANNSTDTVDTVLEKNVTVTHSVNLLEDSHNGKLCGGGGCNTKCQTPLGAINSSLPYQNIHPVTIGECPKYVRSAKLRMVTGLRNTPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 20 - protein, 300 aa, Artificial Sequence
  Synthetic - mini headless HA
MKANLLVLLSALAAADADTICIGYHANNSTDTVDTVLEKNVTVTHSVNLLEGGGGKYVCSAKLRMVTGLRNKPSKQSQGLFGAIAGFTEGGWTGMIDGWYGYHHQNEQGSGYAADQKSTQNAINGITNKVNTVIEKMNIQYTAIGCEYNKSERCMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 21 - protein, 300 aa, Artificial Sequence
  Synthetic - GCN4 headless HA
MKANLLVLLSALAAADADTICIGYHANNSTDTVDTVLEKNVTVTHSVNLLEGGGGKYVCSAKLRMVTGLRNKPSKQSQGLFGAIAGFTEGGWTGMIDGWYGYHHQNEQGSGYAADQKSTQNAINGITNKVNTVIEKMNIQYTAIGCEYNKSERCMKQIEDKIEEIESKIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 22 - protein, 263 aa, Artificial Sequence
  Synthetic - 6SS headless HA
MKANLLVLLSALAAADADTICIGYHANNSTDTVDTVLEKNVTVTHSVNLGSGLRMVTGLRNTPSIQSRGLFGAIAGFIEGGWTGMIDGWYGYHHQNEQGSGYAADQKSTQNAINGITNMVNTVIEKMGSGGSGTDLAELLVLLLNERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 23 - DNA, 792 bp, Artificial Sequence
  Synthetic - 6SS headless HA
atgaaggcaaacctactggtcctgttaagtgcacttgcagctgcagatgcagacacaatatgtataggctaccatgcgaacaattcaaccgacactgttgacacagtactcgagaagaatgtgacagtgacacactctgttaacctgggatcaggcttgaggatggttacaggactaaggaacactccgtccattcaatccagaggtctatttggagccattgccggttttattgaagggggatggactggaatgatagatggatggtatggttatcatcatcagaatgaacagggatcaggctatgcagcggatcaaaaaagcacacaaaatgccattaacgggattacaaacatggtgaacactgttatcgagaaaatgggatcaggcggatcaggcacagatttggcagaattgttagttctactgctaaatgaaaggactctggatttccatgactcaaatgtgaagaatctgtatgagaaagtaaaaagccaattaaagaataatgccaaagaaatcggaaatggatgttttgagttctaccacaagtgtgacaatgaatgcatggaaagtgtaagaaatgggacttatgattatcccaaatattcagaagagtcaaagttgaacagggaaaaggtagatggagtgaaattggaatcaatggggatctatcagattctggcgatctactcaactgtcgccagttcactggtgcttttggtctccctgggggcaatcagtttctggatgtgttctaatggatctttgcagtgcagaatatgcatctga

SEQ ID NO: 24 - protein, 332 aa, Artificial Sequence
  Synthetic - stalk domain of wild-type HA
MKANLLVLLSALAAADADTICIGYHANNSTDTVDTVLEKNVTVTHSVNLLEDSHNGKLNTKCQTPLGAINSSLPYQNIHPVTIGECPKYVRSAKLRMVTGLRNTPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 25 - protein, 263 aa, Artificial Sequence; identical to SEQ ID NO: 22
  Synthetic - stalk domain of 6SS headless HA

SEQ ID NO: 26 - protein, 233 aa, Artificial Sequence
  Synthetic - head domain of wild-type HA
CRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHEC

SEQ ID NO: 27 - protein, 4 aa, Artificial Sequence
  Synthetic - Linker peptide
GGGG

SEQ ID NO: 28 - protein, 6 aa, Artificial Sequence
  Synthetic - Linker peptide
GSGGSG